(19) United States
(12) Patent Application Publication
(10) Pub. No.: US 2018/0136456 A1
(43) Pub. Date: May 17, 2018

(54) ENDOSCOPE SYSTEM

(71) Applicant: OLYMPUS CORPORATION, Tokyo (JP)

(72) Inventors: Tadashi WATANABE, Kamiina-gun (JP); Naoki MATSUO, Suwa-gun (JP); Mamoru SAKASHITA, Okaya-shi (JP)

(73) Assignee: OLYMPUS CORPORATION, Tokyo (JP)

(21) Appl. No.: 15/801,865

(22) Filed: Nov. 2, 2017

Related U.S. Application Data
(63) Continuation of application No. PCT/JP2016/067399, filed on Jun. 10, 2016.

(30) Foreign Application Priority Data
Jun. 30, 2015 (JP) 2015-131913

(51) Int. Cl. G02B 23/26; A61B 1/05; H04N 5/225; G02B 23/24; H04N 7/22

(52) U.S. Cl. CPC G02B 23/26; A61B 1/05; H04N 2005/2255; G02B 23/2484; H04N 7/22; H04N 5/2256

(57) ABSTRACT

An endoscope system includes: an insertion portion in which an image pickup unit configured to pick up an image of an object to be examined and generate a video signal is disposed in a distal end; a video processor configured to process the video signal generated by the image pickup unit; and a signal transmission path connecting the image pickup unit and the video processor, and at least a part of the signal transmission path is a waveguide configured to allow propagation of a millimeter wave or a submillimeter wave, and signal transmission is performed by the waveguide.

### CROSS REFERENCE TO RELATED APPLICATION

This application is a continuation application of PCT/JP2016/067399 filed on Jun. 10, 2016 and claims benefit of Japanese Application No. 2015-131913 filed in Japan on Jun. 30, 2015, the entire contents of which are incorporated herein by this reference.

### BACKGROUND OF THE INVENTION

### 1. Field of the Invention

The present invention relates to an endoscope system and specifically relates to an endoscope system that performs signal transmission via radio waves that propagate through an inside of a waveguide.

### 2. Description of the Related Art

Conventionally, in a medical field and an industrial field, endoscopes each including an image pickup section via which a subject/object is observed have widely been used. Also, a technique in which an endoscope system is configured in such a manner that various signal processing relating to an endoscope is performed by a signal processing apparatus, called a video processor, detachably attached to the endoscope has been known.

As mentioned above, although endoscopes have widely been used as minimally-invasive subject observation means, in recent years, endoscope systems each including what is called a video endoscope in which an image pickup unit including, e.g., an image pickup optical system, an image pickup device and related electric circuits is disposed in a distal end portion of an insertion portion to generate an image signal in the distal end portion of the insertion portion have often been used.

Here, in such a video endoscope system, an image signal generated in the distal end portion of the insertion portion of the endoscope is sent to an image processing section in the video processor through a signal transmission path and an endoscopic image is generated in the image processing section and is provided for observation.

Also, as this type of endoscope, endoscopes each including a flexible insertion portion having an elongated shape, an operation portion connected to a proximal end side of the insertion portion and configured to receive input of various operation signals, and a universal cord, which serves as a signal transmission path that extends from the operation portion and is connected to a video processor in such a manner as above have widely been known.

Then, at the distal end portion of the insertion portion of such an endoscope, a distal end rigid portion with the image pickup device, etc., incorporated is formed, and a bendable bending portion and a long flexible tube portion having flexibility are provided continuously on the proximal end side of the distal end rigid portion.

In conventional video endoscope systems, a form of connection between the image pickup unit and the image processing section via predetermined lead wires to transmit an image signal from the image pickup device such as described in, for example, Japanese Patent Application Laid-Open Publication No. 61-121590 is popular.

However, in recent years, as such method of signal transmission between an image pickup unit and an image processing section, a signal transmission method using optical fiber connection such as indicated in Japanese Patent Application Laid-Open Publication No. 2007-260066 or a signal transmission method using radio waves such as indicated in the description of Japanese Patent No. 5395671 have been proposed.

Also, in recent years, video endoscope systems face an increasing demand for an increase in number of pixels such as represented by what is called high-definition television. Such enhancement in image quality naturally and inevitably leads to an increase in transmission speed of a signal transmitted through a transmission path.

FIG. 11 illustrates a relationship between a transmission distance and a transmission speed in which transmission using electric interconnection (corresponding to the connection using lead wires in Japanese Patent Application Laid-Open Publication No. 61-121590, etc.) is possible, and, for example, indicates that if a transmission distance (length of a transmission path) in a video endoscope system is around 1 to 2 m, a transmission speed of around 2.5 Gbps is a limit in electric interconnection.

In consideration of the communication speed of “2.5 Gbps” substantially corresponding to a transmission speed necessary for practical movie transmission with full high-definition television image quality, it can be seen that with lead wire connection such as indicated in Japanese Patent Application Laid-Open Publication No. 61-121590, movie transmission with an image quality that is equal to or exceeds a full high-definition television image quality is difficult in a video endoscope system.

In other words, the lead wire-used signal transmission method indicated in Japanese Patent Application Laid-Open Publication No. 61-121590 has a problem of failure to respond to an image quality equivalent to full high-definition television due to the transmission speed limit.

Also, as mentioned above, the transmission speed problem in the signal transmission method indicated in Japanese Patent Application Laid-Open Publication No. 61-121590 can be solved by employment of the optical fiber-used signal transmission method (optical interconnection) described in Japanese Patent Application Laid-Open Publication No. 2007-260066.

However, the aforementioned optical fiber-used signal transmission method described in Japanese Patent Application Laid-Open Publication No. 2007-260066 has the following problems.

### 1) Problem Relating to Signal Transmission Reliability

In general, an optical fiber is configured by a single fiber, and thus, a situation such as “an image is suddenly interrupted during use” may occur when the optical fiber is broken because of an effect of, e.g., age.

Here, in the case of lead wire-used connection such as indicated in Japanese Patent Application Laid-Open Publication No. 61-121590, a lead wire is generally configured by a bundle of a plurality of thin wires and the thin wires are gradually broken when the lead wire is broken, and thus, normally, a user can potentially recognize such trouble through, e.g., a flicker in a video image and take a countermeasure such as repair in advance.

### 2) Problem Relating to Manufacturability and Manufacturing Costs

In a normal optical fiber, a tube (core) through which light passes has a diameter of no more than 50 μm, and for positioning for connection, a μm-order accuracy is required. In order to ease such demand, it is possible to use an optical system such as a lens in a connection portion, which, however, increases in size of the connection portion and may cause an increase in manufacturing costs due to an increase in number of components.

### 3) Problem Relating to Communication Circuit Size

In an optical fiber-used system, a need for signal form conversion from an electric signal into an optical signal and from an optical signal to an electric signal causes a need to provide, e.g., a laser diode, a photo diode and drive circuits for the laser diode and the photo diode, which is likely to lead to an increase in circuit size.

In other words, this is because a laser diode and a photo diode are each fabricated in a fabrication process that is different from a fabrication process of a normal IC (integrated circuit) and are less easily housed in a same IC package.

### 4) Problem Relating to Image Pickup Unit Size

Even if signal transmission from an image pickup unit is performed using an optical fiber, it is difficult to replace power transmission and operation clock transmission with optical fiber-used transmission, and thus, in such an optical fiber-used transmission system, it is difficult to eliminate electric connection (lead wire) signal lines from the system.

Also, in addition to the aforementioned problem relating to communication circuit size, it is necessary to further secure an area for lead wire connection (soldering), and thus, an optical fiber-used signal transmission method may cause an increase in size of the image pickup unit, and consequently, an increase in size of the distal end portion of the insertion portion.

Furthermore, video endoscope systems of a type in which an image pickup unit is provided in a distal end rigid portion in a distal end portion of an insertion portion and a bending portion (flexing portion) is provided face a strong need for decreasing a length of the distal end rigid portion as much as possible and thus have a reason to less easily allow an increase in size of the distal end portion of the insertion portion.

On the other hand, the transmission speed problem in the signal transmission method indicated in Japanese Patent Application Laid-Open Publication No. 61-121590 can also be improved by employment of the radio wave-used signal transmission method described in the description of Japanese Patent No. 5395671.

However, the aforementioned radio wave-used signal transmission method described in the description of Japanese Patent No. 5395671 has problems such as indicated below.

### 1) Problem Relating to Signal Transmission Reliability

In general, radio waves are likely to be frequently subjected to various types of electromagnetic interference, and in addition, interruption of signal transmission due to an obstacle on the transmission path may occur, and thus, signal transmission reliability is substantially decreased compared to wired signal transmission.

2) Problem Relating to Signal Transmission from Image Pickup Unit

Also, even where a signal is transmitted using radio waves from the image pickup unit disposed in the distal end portion of the insertion portion, for example, if a body cavity of a subject is observed, only very short-distance communication may be possible as a result of various kinds of electrolytes, water, etc., that are present in the body cavity of the subject impairing propagation of the radio waves.

Therefore, in reality, only the form in which wireless transmission is used only for signal transmission from the operation portion to the image processing section such as described in the description of Japanese Patent No. 5395671 can be employed. In other words, for transmission of signals inside the insertion portion, the form using electric connection (lead wire) signal lines such as indicated in the description of Japanese Patent No. 5395671 is inevitably employed, and thus, the transmission speed restriction is eased (improved), but can hardly be considered as being completely overcome.

### SUMMARY OF THE INVENTION

An endoscope system according to an aspect of the present invention is an endoscope system including: an insertion portion in which an image pickup unit configured to pick up an image of an object to be examined and generate a video signal is disposed in a distal end; a video processing section configured to process the video signal generated by the image pickup unit; and a signal transmission path connecting the image pickup unit and the video processing section, wherein at least a part of the signal transmission path is a waveguide configured to allow propagation of a millimeter wave or a submillimeter wave, and signal transmission is performed by the waveguide.

### BRIEF DESCRIPTION OF THE DRAWINGS

FIG. 1 is a perspective view illustrating a schematic configuration of an endoscope system according to a first embodiment of the present invention;

FIG. 2 is a block diagram illustrating a functional configuration of a major part of the endoscope system according to the first embodiment;

FIG. 3 is a major part perspective view for describing, e.g., shape of a waveguide employed in the endoscope system according to the first embodiment where it is assumed that the waveguide is a round waveguide tube;

FIG. 4 is a diagram indicating electric and magnetic field distributions and a cutoff wavelength in a TE₁₁ mode used as a power feed line for an antenna in the waveguide employed in the endoscope system according to the first embodiment;

FIG. 5 is a diagram indicating electric and magnetic field distributions and a cutoff wavelength in a TE₀₁ mode, which is drawing attention as a low-loss millimeter wave transmission line in the waveguide employed in the endoscope system according to the first embodiment;

FIG. 6 is an enlarged major part perspective view illustrating a structure of an image pickup unit and the waveguide in the endoscope system according to the first embodiment;

FIG. 7 is an enlarged major part perspective view illustrating the structure of the image pickup unit and the waveguide in the endoscope system according to the first embodiment partly in cross-section;

FIG. 8 is a block diagram illustrating a functional configuration of a major part of an endoscope system according to a second embodiment of the present invention;

FIG. 9 is a block diagram illustrating a functional configuration of a major part of an endoscope system according to a third embodiment of the present invention;

FIG. 10 is a block diagram illustrating a functional configuration of a major part of an endoscope system according to a fourth embodiment of the present invention;

FIG. 11 is a model chart indicating a relationship between possible transmission distance and transmission speed in transmission via electric interconnection used in a conventional endoscope system;

FIG. 12 is a block diagram illustrating a functional configuration of a major part of an endoscope system according to a fifth embodiment of the present invention;

FIG. 13 is a diagram illustrating a dielectric loss of a dielectric material in a waveguide tube employed in the endoscope system according to the fifth embodiment;

FIG. 14 is a diagram indicating results of simulations of a dielectric loss of a dielectric material in a waveguide tube employed in the endoscope system according to the fifth embodiment;

FIG. 15 is an enlarged view illustrating a major part of the results of simulations of a dielectric loss of a dielectric material in a waveguide tube employed in the endoscope system according to the fifth embodiment;

FIG. 16 is a cross-sectional view illustrating a cross-section of the waveguide tube employed in the endoscope system according to the fifth embodiment;

FIG. 17 is an enlarged view illustrating a major part of the cross-section of the waveguide tube employed in the endoscope system according to the fifth embodiment;

FIG. 18 is a block diagram illustrating a functional configuration of a major part of an endoscope system according to a sixth embodiment of the present invention;

FIG. 19 is a cross-sectional view illustrating a cross-section of a waveguide tube employed in the endoscope system according to the sixth embodiment;

FIG. 20 is a diagram indicating results of simulations of a dielectric loss of dielectric materials in a waveguide tube employed in the endoscope system according to the sixth embodiment; and

FIG. 21 is a cross-sectional view illustrating a cross-section of a modification of the waveguide tube employed in the endoscope system according to the sixth embodiment.

### DETAILED DESCRIPTION OF THE PREFERRED EMBODIMENTS

Embodiments of the present invention will be described below with reference to the drawings.

Also, these embodiments are not intended to limit the present invention. Furthermore, in the statements in the drawings, parts that are identical to each other are provided with a same reference numeral. Furthermore, it should be noted that the drawings are schematic ones and, e.g., a relationship between a thickness and a width of each member and ratios among the respective members are different from actual ones. Also, parts that are different in dimensional relationship and ratio depending on the drawings are included in the drawings.

### First Embodiment

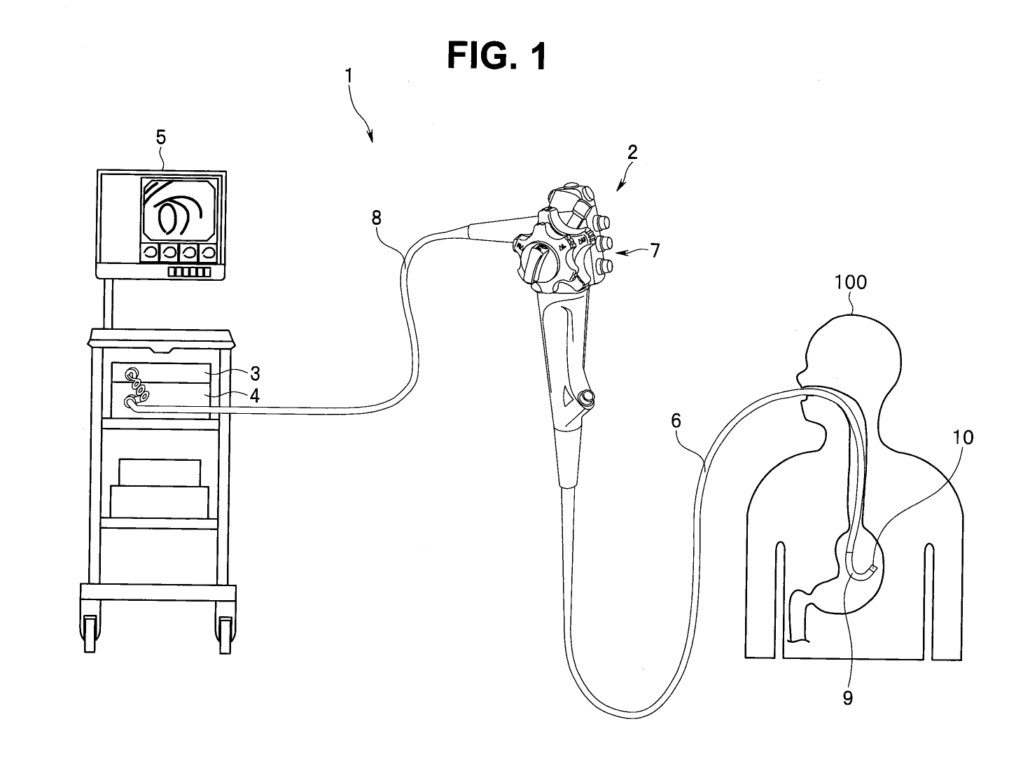

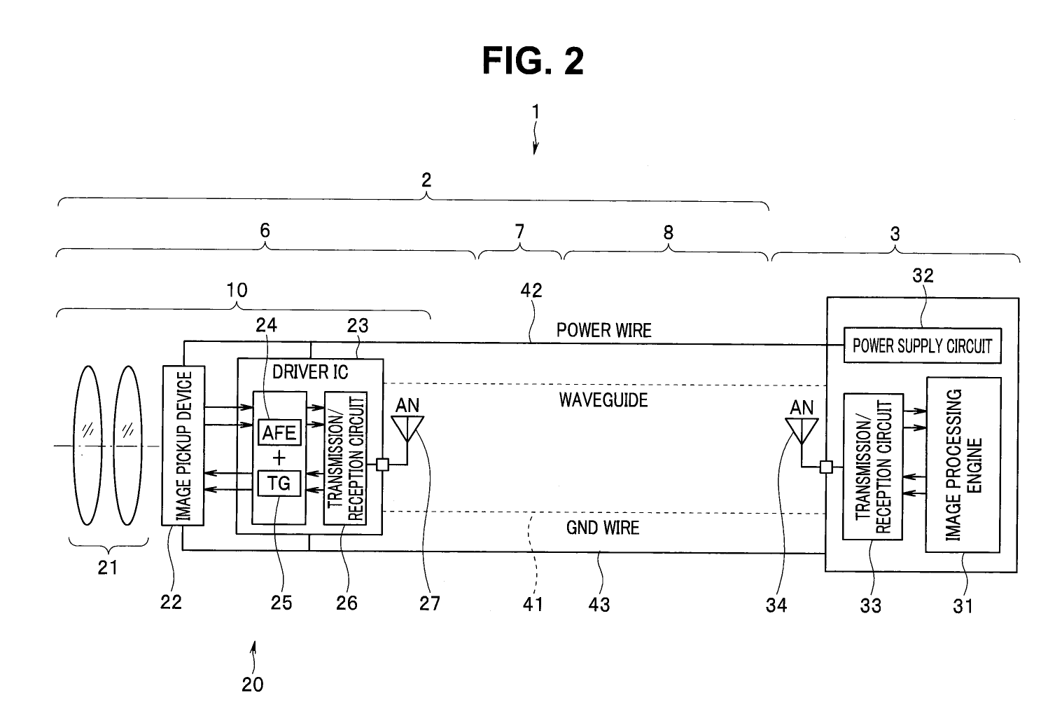

FIG. 1 is a perspective view illustrating a schematic configuration of an endoscope system according to a first embodiment of the present invention, and FIG. 2 is a block diagram illustrating a functional configuration of a major part of the endoscope system according to the first embodiment.

As illustrated in FIG. 1, an endoscope system 1 is what is called an endoscope system for upper digestive tract, and mainly includes: an endoscope 2 including an image pickup section configured to, upon insertion of a distal end portion of the endoscope 2 to a body cavity of a subject 100, pick up an image of the inside of the body of the subject 100 and output an image signal of an object image; a video processor 3 including an image processing section configured to perform predetermined image processing of the image signal outputted from the image pickup section in the endoscope 2, a video processor 3 being configured to comprehensively control operation of the entire endoscope system 1; a light source apparatus 4 configured to generate illuminating light to be outputted from a distal end of the endoscope 2; and a display apparatus 5 configured to display an image resulting from the image processing in the video processor 3.

The endoscope 2 includes: an insertion portion 6 including the image pickup section in the distal end portion, the insertion portion 6 being mainly configured by an elongated shape portion having flexibility; an operation portion 7 connected to the proximal end side of the insertion portion 6, the operation portion 7 being configured to receive input of various operation signals; and a universal cord 8 extending from the operation portion 7 toward the proximal end side and connecting with the video processor 3 and the light source apparatus 4.

Here, the endoscope 2 includes a signal transmission path for transmission of, e.g., an image signal from the image pickup section, the signal transmission path being provided so as to extend from the image pickup section in the insertion portion 6 to the image processing section in the video processor 3 via the inside of each of the insertion portion 6, the operation portion 7 and the universal cord 8, between the image pickup section disposed in the distal end portion of the insertion portion 6 and the image processing section in the video processor 3.

Then, in the endoscope system according to the present embodiment, the signal transmission path is configured by a waveguide that lets millimeter waves or submillimeter waves (hereinafter, may representatively be referred to as millimeter waves) through (the “waveguide” will be described in detail later).

Referring back to FIG. 1, the insertion portion 6 includes: a distal end rigid portion 10 disposed at a most distal end portion, in which, e.g., an image pickup device 22 included in the image pickup section is incorporated; a bendable bending portion 9 that is disposed on the proximal end side of the distal end rigid portion 10 and includes a plurality of bending pieces; and a long flexible tube portion 11 having flexibility, the flexible tube portion 11 being connected to the proximal end side of the bending portion 9.

Also, as illustrated in FIG. 2, in the present embodiment, in the distal end rigid portion 10 disposed at a most distal end of the insertion portion 6, an image pickup unit 20 including, e.g., the image pickup device 22 configured to pick up a subject image and output a predetermined image signal by means of photoelectric conversion is disposed.

The image pickup unit 20 includes: an image pickup optical system 21 configured to allow entrance of a subject image; the image pickup device 22 provided at an image forming position for the image pickup optical system 21, the image pickup device 22 being configured to receive light collected by the image pickup optical system 21 and perform photoelectric conversion of the light to an electric signal; a driver IC 23 disposed on the adjacent proximal end side of the image pickup device 22 and configured to drive the image pickup device 22 and perform predetermined processing on an image pickup signal outputted from the image pickup device 22; and a transmission/reception antenna 27 (which will be described in detail later) for signal transmission/reception via a waveguide 41 (which will be described in detail later), the transmission/reception antenna 27 being provided on the proximal end side of the driver IC 23.

For the image pickup device 22, in the present embodiment, a CMOS (complementary metal oxide semiconductor) image sensor having a pixel count of no less than 2 million pixels, which is a pixel count corresponding to or exceeding a pixel count for what is called full high-definition television is employed.

The driver IC 23 includes: an analog front end (AFE) 24 configured to perform denoising and A/D conversion of an electric signal outputted by the image pickup device 22; a timing generator (TG) 25 configured to generate pulses for a driving timing for the image pickup device 22 and various signal processing in, e.g., the AFE 24; a transmission/reception circuit 26 for transmission/reception of the digital signal outputted by the AFE 24 to/from the image processing section in the video processor 3 via the waveguide 41, the transmission/reception antenna 27 being connected to the transmission/reception circuit 26; and a non-illustrated control section configured to control operation of the image pickup device 24.

The transmission/reception circuit 26 is a millimeter wave/submillimeter wave communication circuit formed by what is called an MMIC (monolithic microwave integrated circuit).

Also, in the present embodiment, the respective circuits such as the analog front end AFE 24, the timing generator TG 25 and the transmission/reception circuit 26 of the driver IC 23 are each created through a silicon CMOS process and are sufficiently downsized.

Also, the image pickup device 22 and the driver IC 23 are connected via a ceramic substrate, and also, a plurality of passive components such as capacitors are mounted on the ceramic substrate (which will be described in detail later).

On the other hand, the video processor 3 includes: an image processing engine 31, which serves as the image processing section configured to perform predetermined image processing of an image signal outputted from the image pickup unit 20 in the endoscope 2; a power supply circuit 32 configured to produce power to be supplied to, e.g., the image pickup device 22 in the endoscope 2; a transmission/reception circuit 33 for transmission/reception of a predetermined signal to/from the image pickup unit 20 in the endoscope 2 via the waveguide; and a transmission/reception antenna 34 connected to the transmission/reception circuit 33.

Here, as with the transmission/reception circuit 26, the transmission/reception circuit 33 in the video processor 3 is also formed by what is called an MMIC (monolithic microwave integrated circuit).

Here, as illustrated in FIG. 2, as described above, the waveguide 41 is provided as the signal transmission path inside the insertion portion 6, the operation portion 7 and the universal cord 8 of the endoscope 2, and inside the universal cord 8, etc., a power wire 42 and a ground wire (GND wire) 43 for power supplied from the power supply circuit 32 in the video processor 3 are disposed in parallel with the waveguide 41.

Then, the image pickup device 22 and the respective circuits in the driver IC 23 in the endoscope 2 are supplied with power from the power supply circuit 32 of the video processor 3 via the power wire 42 and the ground wire (GND wire) 43.

### <Configuration of Waveguide and Transmission/Reception Circuit and Image Pickup Unit>

Next, the waveguide and the transmission/reception circuits, and peripheral circuits (e.g., the image pickup unit) of the waveguide and the transmission/reception circuits that characterize the endoscope system according to the present embodiment will be described in detail.

As described above, the present invention proposes a new signal transmission method using a waveguide configured to allow transmission of millimeter waves or submillimeter waves (radio waves having a frequency of approximately 30 to 600 GHz) instead of a lead wire-used signal transmission method and an optical fiber-used signal transmission method, which have conventionally been used as signal transmission methods connecting an image pickup section in an endoscope and an image processing section in a video processor.

In the present embodiment, millimeter waves and submillimeter waves refer to waves having a wavelength on the order of from millimeters to submillimeters (around 0.5 to 10 mm).

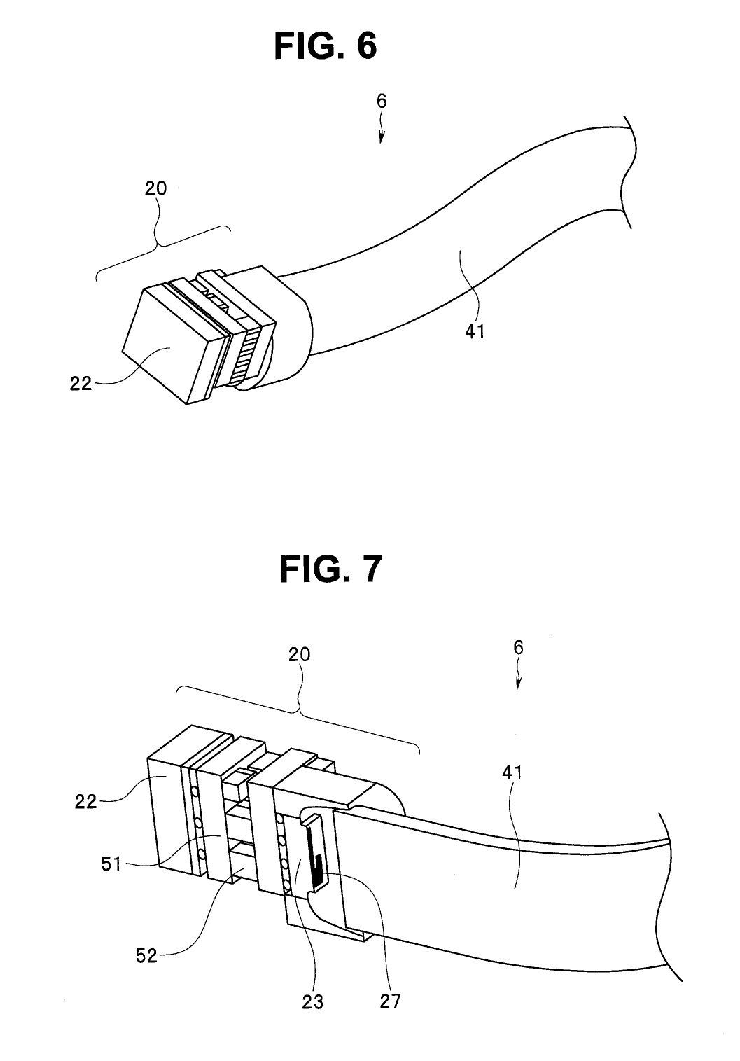

FIG. 6 is an enlarged major part perspective view illustrating a structure of the image pickup unit and the waveguide in the endoscope system according to the first embodiment, and FIG. 7 is an enlarged major part perspective view illustrating the structure of the image pickup unit and the waveguide in the endoscope system partly in cross-section. Note that in FIGS. 6 and 7, the image pickup optical system in the image pickup unit is omitted.

As illustrated in FIGS. 6 and 7, in the image pickup unit 20, the driver IC 23 is disposed via a ceramic substrate 51 on the proximal end side of the image pickup device 22 provided at the image forming position for the non-illustrated image pickup optical system 21. Here, a plurality of passive components such as a capacitor 52 are mounted on the ceramic substrate 51.

Also, as illustrated in FIG. 7, a distal end portion of the waveguide 41 configured to allow transmission of millimeter waves or submillimeter waves is connected to the proximal end side of the driver IC 23 with the transmission/reception antenna 27 interposed between the driver IC 23 and the distal end portion of the waveguide 41, the transmission/reception antenna 27 being integrated with the package of the driver IC 23.

The waveguide 41 is made to extend toward the proximal end side of the insertion portion 6 after the distal end side of the waveguide 41 is connected to the driver IC 23 disposed in the distal end rigid portion 10. For more detail, the waveguide 41 is inserted in a part, on the proximal end side relative to the driver IC 23, of the insertion portion 6, that is, an inner portion of the insertion portion 6 including a part, on the proximal end side portion relative to a site of disposition of the driver IC 23, of the distal end rigid portion 10 as well as the bending portion 9 and the flexible tube portion 11 on the more proximal end side and then is disposed so as to reach a position of the video processor 3 through the inside of the operation portion 7 and the inside of the universal cord 8.

Note that the proximal end side of the waveguide 41 may be connected to the video processor 3 through conversion in a connector provided at an end of the universal cord 8.

Also, in the present embodiment, the waveguide 41 is formed by providing metal plating to the periphery of a polystyrene resin (dielectric material having a permittivity of approximately 2.3 and a dielectric tangent of approximately 0.0002). Also, in the present embodiment, an inner diameter of the metal plating surface in the waveguide 41 is set as 1.4 mm and a frequency of radio waves used for transmission of image information is set as approximately 180 GHz (wavelength in the waveguide is set as approximately 1.1 mm).

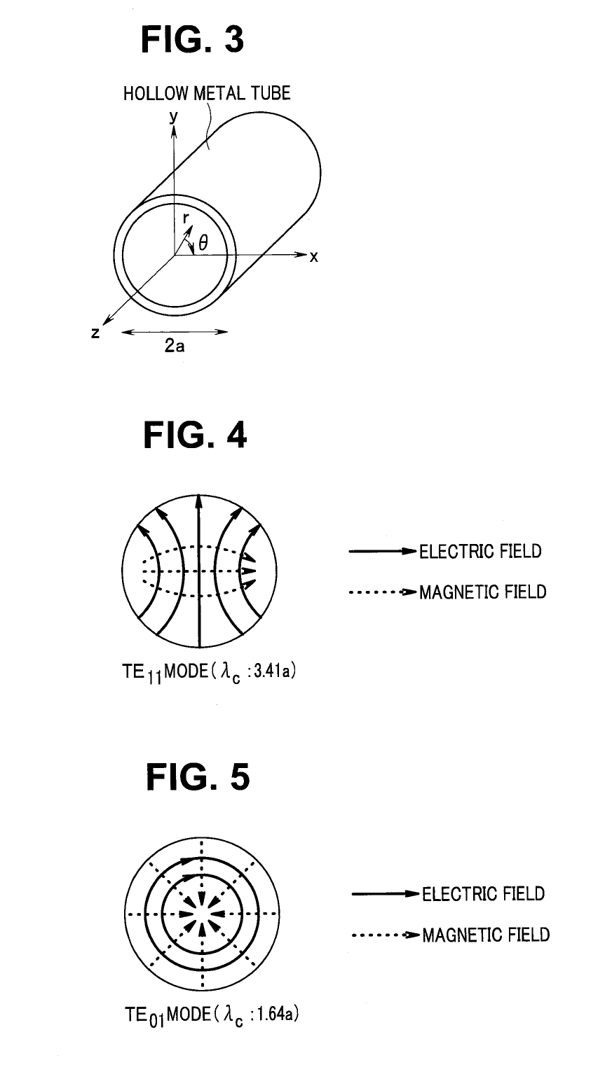

Here, for effective utilization of the configuration of the present invention, it is necessary to properly select a shape and a dimension of the waveguide 41, and the shape and the dimension are highly related with wavelength of radio waves used. The shape and the dimension of the waveguide 41 in the present embodiment will be described below with reference to FIGS. 3 to 5 on the assumption that a round waveguide tube is used as a waveguide configured to allow transmission of millimeter waves or submillimeter waves.

FIG. 3 is a major part perspective view for describing, e.g., a shape of waveguide employed in the endoscope system according to the first embodiment where it is assumed that the waveguide is a round waveguide tube, FIG. 4 is a diagram indicating electric and magnetic field distributions and a cutoff wavelength in a TE₁₁ mode used as a power feed line for an antenna in the waveguide employed in the endoscope system according to the first embodiment, and FIG. 5 is a diagram indicating electric and magnetic field distributions and a cutoff wavelength in a TE₀₁ mode, which is drawing attention as a low-loss millimeter wave transmission line in the waveguide employed in the endoscope system according to the first embodiment.

Although FIG. 3 includes the statement “hollow metal tube”, the “hollow metal tube” is a mere example for clearly indicating operation of the invention and is not intended to limit the embodiment of the present invention. More specifically, as means for providing a waveguide, any of various forms such as a form obtained by, e.g., providing metal conductor plating to the inside of a flexible resin conduit and forms using a square waveguide tube or a round or square dielectric material waveguide may be employed, and each of such forms can provide effects of the present invention.

In the case of a round waveguide tube assumed here, a plurality of modes of transmitted waves may exist. The plurality of modes are roughly divided into TE modes and TM modes and further divided into θ-direction and r-direction mode numbers.

FIGS. 4 and 5 illustrate overviews of electric and magnetic field distributions in a TE₁₁ mode used as a power feed line for an antenna and a TE₀₁ mode, which is drawing attention as a low-loss millimeter wave transmission line, and cutoff wavelengths λ_{C} in the TE₁₁ mode and the TE₀₁ mode.

A waveguide tube cannot transmit radio waves having a wavelength that is equal to or exceeding a certain wavelength because of a structure of the waveguide tube, and the cutoff wavelength λ_{C} indicates a wavelength that “radio waves equal to or exceeding the wavelength cannot be transmitted”. Here, since the TE₁₁ mode has a longest wavelength, it can be seen that a wavelength that is equal to or exceeding λ_{C} in the TE₁₁ mode (where a is an inner radius of the tube, λ_{C}=3.41×a) cannot be transmitted.

A relationship between a dimension and a cutoff wavelength indicated here exists also in, e.g., a square waveguide tube and a dielectric material-used waveguide, and the above description is not intended to limit the form of the waveguide.

In general, many endoscopes include an insertion portion and a universal cord having an outer dimension of around no more than 10 mm because of purposes of the insertion portion and the universal cord and some endoscopes include an insertion portion and a universal cord having an even smaller diameter; however, if it is assumed that a signal transmission line extending through the insertion portion and the universal cord has a cylindrical shape, the signal transmission line desirably has a diameter of around no more than ϕ6 mm.

Then, from the aforementioned cutoff wavelength λ_{C} calculation expression, λ_{C} of a hollow metal conduit having an inner diameter of ϕ6 mm is 10.23 mm; however, λ_{C} indicates a limit of a wavelength that can be transmitted and thus, a shorter wavelength of a signal to be transmitted is more desirable, and in the present invention, it is assumed that an effective wavelength range is a range of no more than 10 mm (equal to or below a millimeter wave=a frequency range of no less than 30 GHz).

On the other hand, a communication line inside an endoscope is required to be light and thin, but if the communication line is too thin, the problem in manufacturability or manufacturing costs may occur, and as already described, such thin communication line affects signal transmission reliability.

More specifically, in the aforementioned optical fiber described in Japanese Patent Application Laid-Open Publication No. 2007-260066, a core diameter is small, that is, around 10 μm in the case of a single mode fiber, and around 50 μm even in the case of a multimode fiber; however, according to the embodiment of the present invention, a signal transmission line (waveguide) that is much thicker than the optical fibers can be used, and thus, the problem in manufacturability and manufacturing costs and the problem relating to signal transmission reliability can be solved.

In consideration of such circumstances, it is desirable that a diameter of a waveguide be desirably no less than 0.2 mm; however, the value is not absolute.

In addition, a wavelength of radio waves used for signal transmission being short provides a plurality of advantages such as ease of information density enhancement and ease of transmission/reception circuit downsizing. However, a millimeter wave/submillimeter wave band is difficult to handle because of the short wavelengths and has a problem in that as the wavelength is shorter, the efficiency of the circuit is decreased.

Therefore, until recent years, use of millimeter wave/submillimeter wave bands was not popular, but as a result of advancement of millimeter wave/submillimeter wave utilization techniques along with advancement of microcircuit techniques using semiconductor processes, and advancement of countermeasures against the circuit efficiency decrease, an environment in which even the submillimeter wave band can easily be used is becoming established over the past several years.

As a result of the present applicant conducting a diligent study comprehensively in consideration of such circumstances, it was found that use of radio waves for signal transmission inside an endoscope is advantageous if the radio waves have a frequency of up to around 600 GHz.

Radio waves of 600 GHz have a wavelength of around 0.5 mm in a vacuum, but can have a shorter wavelength inside a dielectric material, and thus, enable transmission using a waveguide of around ϕ0.2 mm and do not conflict with the aforementioned solutions to the problem relating to manufacturability and manufacturing costs and the problem relating to signal transmission reliability.

As already described, the scope of effective utilization of the present invention is related to the shape and the dimension of the waveguide, and in reality, various shapes can be contemplated as the shape of the waveguide, and thus, it is difficult to express the essence and the effective scope with definitions according to the shape and dimension.

Therefore, in the present embodiment, as described above, in consideration of the clear relationship between the shape and dimension of the waveguide and the wavelength of radio waves used, a constituent feature of the invention is determined according to the wavelength of radio waves used.

### (Operation)

Next, operation of the endoscope system according to the present embodiment configured as described above will be described.

The image pickup device 22 receives a subject image entering the image pickup optical system 21, via an image pickup device surface and performs photoelectric conversion of the image into electric signals and outputs the electric signals as analog image pickup signals.

Subsequently, the driver IC 23 performs predetermined processing, such as A/D conversion, of the analog image pickup signals outputted from the image pickup device 22, in the AFE 24 inside the driver IC 23 and outputs the resulting digital image signal. Note that the image pickup signals are converted into a serial digital signal in a non-illustrated parallel/serial conversion section.

Furthermore, the driver IC 23 modulates millimeter/submillimeter carrier waves according to the image signal in the transmission/reception circuit 26 configured by an MMIC and sends the millimeter/submillimeter radio waves with the relevant image information included, from the transmission/reception antenna 27 toward the waveguide 41.

Subsequently, the millimeter/submillimeter waves sent from the transmission/reception antenna 27 is sent to the video processor 3 through the waveguide 41 (which is, as described above, disposed inside the part, on the proximal end side relative to the driver IC 23 disposed in the distal end rigid portion 10, of the insertion portion 6, the bending portion 9 and the flexible tube portion 11 on the proximal end side of the part and the inside of the operation portion 7 and inside the universal cord 8).

The millimeter waves (millimeter waves with the image information included) sent inside the waveguide 41 is received by the transmission/reception antenna 34 in the video processor 3.

Subsequently, predetermined image information is extracted from the millimeter waves (millimeter waves with the image information included) received by the transmission/reception antenna 34, by the transmission/reception circuit 33 in the video processor 3.

Then, the image information extracted by the transmission/reception circuit 33 is subjected to image processing in the image processing engine 31 as appropriate and is projected on the display apparatus 5.

### (Effects)

As described above, the present first embodiment enables highly-reliable signal transmission through a wired millimeter wave communication path (waveguide), and in terms of a transmission speed of image information, enables a high-definition image largely exceeding full high-definition television to be sent at a practical frame rate.

Here, where the waveguide 41 in the present embodiment has a millimeter-order thickness and the transmission/reception antenna 27 and the transmission/reception antenna 34 are present within a dimension range of the waveguide 41, efficient communication can be performed, and thus the waveguide and the antennas can easily be connected.

Also, as described above, each of the analog front end section, the timing generator section and the transmission/reception circuit in the driver IC 23 configured to process image information from the image pickup device 22 and perform signal transmission is fabricated by a silicon CMOS process and thus sufficiently downsized.

From among the circuits and sections, the transmission/reception circuit 26 and the transmission/reception circuit 33 are each configured by a monolithic microwave integrated circuit (MMIC) and thus contribute to the circuit downsizing.

As described above, as a result of the downsizing of the driver IC 23, highly-reliable transmission of a full high-definition television image signal and downsizing of the distal end portion are both enabled.

Furthermore, as a result of use of a waveguide tube, radio waves emitted from the image pickup unit-side antenna propagate in such a manner that the radio waves are enclosed in the waveguide tube, minimizing loss due to, e.g., diffusion. In other words, minimization of an amount of power required for transmission can also be achieved.

Also, effects of the present embodiment will be described in comparison with the techniques described in Japanese Patent Application Laid-Open Publication No. 61-121590, Japanese Patent Application Laid-Open Publication No. 2007-260066 and the description of Japanese Patent No. 5395671, which are the conventional techniques stated above.

As described above, in the endoscope system according to the present embodiment, the millimeter waves/submillimeter waves transmitted through the waveguide 41 are radio waves having a wavelength on the order of millimeters to submillimeters and have a frequency of roughly 30 to 600 GHz, and thus, the aforementioned transmission speed problem in the lead wire method described in Japanese Patent Application Laid-Open Publication No. 61-121590 can be solved. In other words, a signal transmission speed of no less than 2.5 Gbps can be achieved without difficulty.

Also, millimeter waves/submillimeter waves have advantages of ease of using various signal modulation methods proven in normal radio wave communications and ease of information transmission density enhancement, and thus, depending on the configuration of the device, the information speed can be enhanced relative to the aforementioned optical fiber-used signal transmission method described in Japanese Patent Application Laid-Open Publication No. 2007-260066.

Furthermore, the endoscope system according to the present embodiment enables solution of the problem relating to signal transmission reliability in Japanese Patent Application Laid-Open Publication No. 2007-260066 and the description of Japanese Patent No. 5395671.

In other words, first, millimeter waves/submillimeter waves, which form a transmitted signal, are transmitted in such a manner that the millimeter wave/submillimeter waves are enclosed in a waveguide having good transmission efficiency, and thus, sufficient signal strength can be obtained and transmission is prevented from becoming unstable along the path.

Also, even if the waveguide deteriorates with age and is broken because of, e.g., cracking, millimeter waves/submillimeter waves propagate also through the broken part, and for the aforementioned reason, in the present embodiment, the signal transmission path (waveguide) can be made to be sufficiently thick, and thus, signal transmission may deteriorate in quality, but cannot suddenly be interrupted.

In addition, in the optical fiber-used signal transmission in Japanese Patent Application Laid-Open Publication No. 2007-260066, signal transmission is highly likely to be interrupted where the signal transmission path (core part) is no more than around ϕ50 μm, which is extremely small.

Also, as described above, in the present embodiment, as the image pickup device 22, a solid-state image pickup device including a number of pixels equal to or exceeding two million pixels, that is, corresponding to or exceeding a number of pixels for what is called full high-definition television including is employed, but as described above, the present embodiment enables achievement of a signal transmission speed of no less than 2.5 Gbps, and thus, causes no trouble even with such pixel count.

Furthermore, in the present embodiment, as each of the transmission/reception circuit 26 and the transmission/reception circuit 33, a millimeter wave/submillimeter wave communication circuit configured by an MMIC (monolithic microwave integrated circuit) is employed, and thus, the communication circuits can be downsized and the problem relating to Japanese Patent Application Laid-Open Publication No. 2007-260066 can be solved.

Furthermore, the endoscope system according to the present embodiment includes the endoscope 2 including, as an element, the insertion portion 6 including the bending portion and the flexible tube portion, that is, a video endoscope system having, as a result of a flexing portion being included, a function that freely changes a direction of the distal end rigid portion and acquires an image in a desired direction also solves the problem relating image pickup unit size in Japanese Patent Application Laid-Open Publication No. 2007-260066 and enables provision of a video endoscope system including a small distal end portion.

This is because not only use of millimeter waves/submillimeter waves enable provision of a small communication circuit but also use of millimeter waves/submillimeter waves enable power source energy and operation clocks to be sent to an image pickup unit from the outside of the image pickup unit.

In other words, full high-definition television image signal transmission, which is difficult in an optical fiber-used signal transmission method such as indicated in Japanese Patent Application Laid-Open Publication No. 2007-260066 and downsizing of the distal end portion can be both achieved.

As described above, video endoscope systems of the type face a strong need for reduction in size of the distal end portion (non-flexing part) and thus can actually greatly contribute to enhancement in function of an endoscope such as freely performing observation in a narrow space.

Although the endoscope system according to the present embodiment is premised on the assumption that the endoscope system is a video endoscope system for upper digestive tract, the endoscope system according to the present embodiment can provide effects that are similar to the above regardless of the type of the endoscope as long as the endoscope system is a video endoscope system including an insertion portion with an image pickup unit disposed in a distal end portion, an image processing section configured to process an image signal generated by the image pickup unit, and a signal transmission path connecting the image pickup unit and the image processing section.

In other words, effects that are similar to the above can be provided by each of, e.g., various endoscopes for digestive tract such as endoscopes for lower digestive tract (large intestine) as well as various surgical endoscopes used in endoscopic surgery and various industrial endoscopes for observing the inside of a pipe, a machine or various structural objects.

Also, although in the present embodiment, as described above, as the configuration of the image pickup unit 20, the configuration including the image pickup optical system 21, image pickup device 22, the driver IC 23, the transmission/reception antenna 27 and the non-illustrated capacitors, the driver IC 23 including the analog front end (AFE) section 24, the timing generator (TG) section 25 and the transmission/reception circuit 26 is employed, effects that are similar to the above can be provided even with a configuration that is not such configuration.

For example, the analog front end (AFE) section 24 and the timing generator (TG) section 25 included in the driver IC 23 can be included in the image pickup device 22, and in this case, also, effects that are similar to the above can be provided.

Also, although each of the transmission/reception circuit 26 on the endoscope 2 side and the transmission/reception circuit 33 on the video processor 3 side is a monolithic microwave integrated circuit (MMIC) and thus, as described above has a configuration that is optimum for downsizing the circuit, even if no monolithic microwave integrated circuits are used, a full high-definition television image signal can be transmitted with high reliability and effects that are similar to the above can be obtained.

### Second Embodiment

Next, a second embodiment of the present invention will be described.

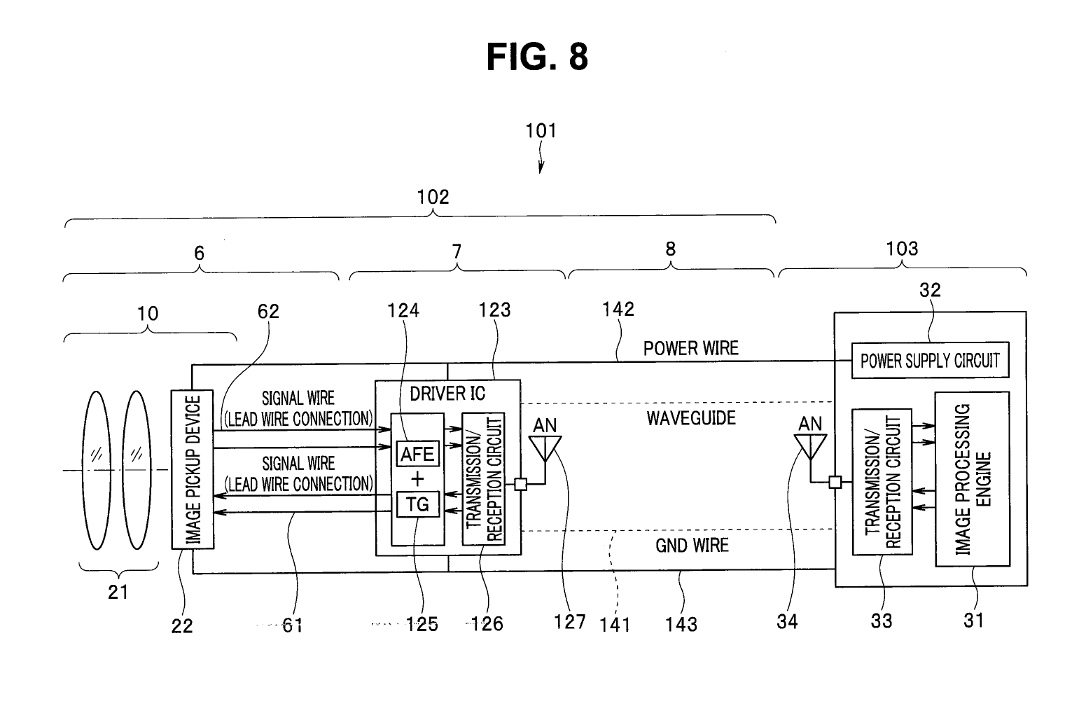

FIG. 8 is a block diagram illustrating a functional configuration of a major part of an endoscope system according to the second embodiment of the present invention.

The endoscope system according to the present second embodiment is basically similar in configuration to the endoscope system according to the first embodiment, and thus, here, only differences from the first embodiment will be described and detailed description of the rest will be omitted.

As described above, in the endoscope system 1 according to the first embodiment, the driver IC 23 that serves to drive the image pickup device 22 and perform predetermined processing of image pickup signals outputted from the image pickup device 22 and send the resulting millimeter/submillimeter radio waves to the waveguide 41 is disposed in the distal end rigid portion 10 of the insertion portion 6, and the waveguide 41 is provided so as to extend inside a part, on the proximal end side relative to the distal end rigid portion 10, of the insertion portion 6, the operation portion 7 and the universal cord 8.

On the other hand, in an endoscope system 101 according to the present second embodiment, a driver IC that serves as described above is provided in an operation portion 7, and a waveguide that serves as in the first embodiment is provided so as to extend inside the operation portion 7 and a universal cord 8.

As illustrated in FIG. 8, as in the first embodiment, the endoscope system 101 according to the present second embodiment is what is called an endoscope system for upper digestive tract mainly including: an endoscope 102 including an image pickup section configured to pick up an image of the inside of the body of a subject and outputs an image signal of the object image; a video processor 103 including an image processing section configured to perform predetermined image processing of the image signal outputted from the image pickup section in the endoscope 102, the video processor 103 being configured to comprehensively control operation of the entire endoscope system 101, and a non-illustrated light source apparatus and a display apparatus.

In the present second embodiment, the endoscope 102 includes an insertion portion 6, an operation portion 7 and a universal cord 8 that are similar to the insertion portion 6, the operation portion 7 and the universal cord 8 in the first embodiment, and the insertion portion 6 includes a distal end rigid portion 10 with, e.g., an image pickup device 22 incorporated.

In the present second embodiment, as illustrated in FIG. 8, a driver IC 123 that serves as with the driver IC 23 in the first embodiment is provided in the operation portion 7.

The driver IC 123 includes an analog front end (AFE) 124 and a timing generator (TG) 125, which serve as in the first embodiment, and the AFE 124 and the TG 125 are connected to the image pickup device 22 via signal wires (lead wire) 61, 62 inserted in the insertion portion 6.

Note that the signal wires (lead wires) 61, 62 each have a length of approximately 80 cm in the present embodiment.

In other words, in the present second embodiment, the image pickup device 22 and the driver IC 123 are connected via the lead wires of approximately 80 cm.

Furthermore, the driver IC 123 includes a transmission/reception circuit 126 and a transmission/reception antenna 127 each having a configuration that is similar to the relevant configuration in the first embodiment.

Also, a distal end portion of a waveguide 141 that is similar to the waveguide in the first embodiment, the waveguide 141 being configured to allow transmission of millimeter waves or submillimeter waves, is connected to the proximal end side of the driver IC 123.

In the present second embodiment, the waveguide 141 is arranged in such a manner that the distal end side of the waveguide 141 is connected to the driver IC 23 disposed in the operation portion 7 and then is disposed so as to reach a position of the video processor 103 through the inside of the universal cord 8.

On the other hand, the video processor 103 includes an image processing engine 31, a power supply circuit 32, a transmission/reception circuit 33 and a transmission/reception antenna 34 that are similar to the image processing engine 31, the power supply circuit 32, the transmission/reception circuit 33 and the transmission/reception antenna in the first embodiment.

Also, as illustrated in FIG. 8, in the present second embodiment, inside the operation portion 7 and the universal cord 8 in the endoscope 102, the waveguide 141 is provided as a signal transmission path, and inside the universal cord 8, etc., a power wire 142 and a ground wire (GND wire) 143 for power supplied from the power supply circuit 32 in the video processor 103 are provided in parallel with the waveguide 141 and the above-described signal wires 61, 62.

As described above, in the present second embodiment, a signal transmission path from the image pickup device 22 to the driver IC 123 disposed inside the operation portion 7 is provided by the signal wires using lead wire connection (electric connection) and a signal transmission path from the driver IC 123 inside the operation portion 7 to the image processing section in the video processor 103 is provided by the waveguide 141 configured to allow propagation of millimeter waves, and signal transmission is performed by the signal transmission paths.

Here, the lead wires connecting the image pickup device 22 and the driver IC 123 each have a length of approximately 80 cm and thus enable conveyance of a full high-definition television image signal.

As described above, as in the first embodiment, the present second embodiment enables highly-reliable signal transmission through a wired millimeter wave communication path (waveguide), and, in terms of image information transmission speed, enables a high-definition image for up to around full high-definition television to be sent at a practical frame rate. Also, for other effects, effects that are similar to the effects of the first embodiment can be exerted.

### Third Embodiment

Next, a third embodiment of the present invention will be described.

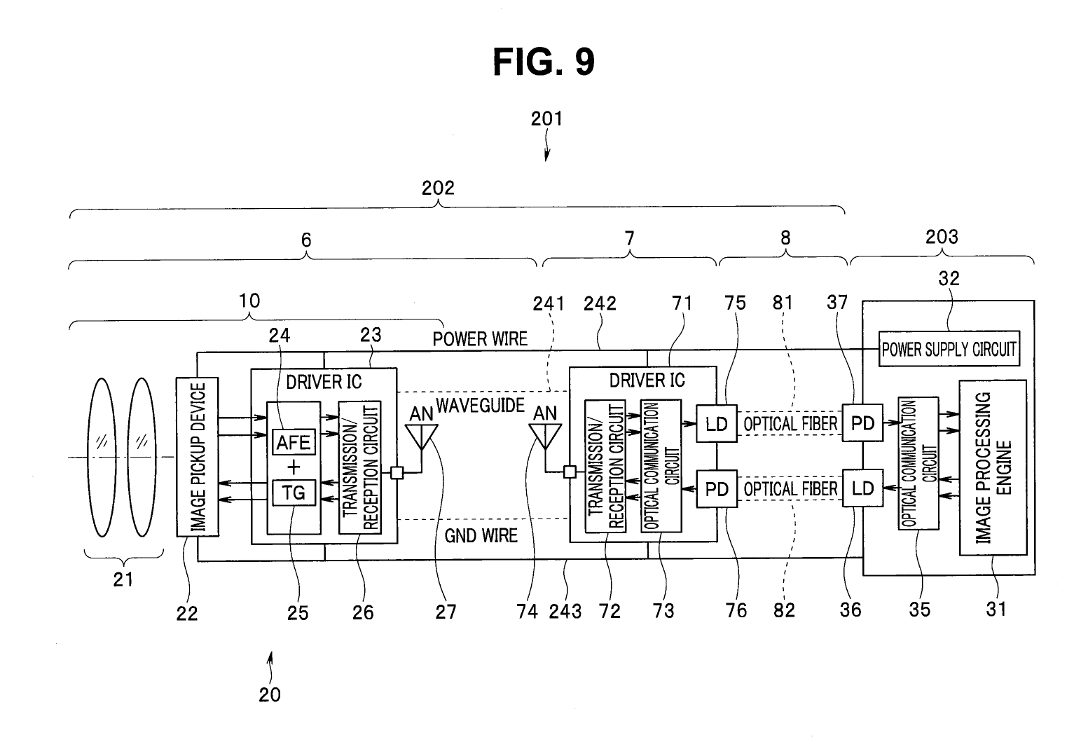

FIG. 9 is a block diagram illustrating a functional configuration of a major part of an endoscope system according to the third embodiment of the present invention.

The endoscope system according to the present third embodiment is similar in configuration to the endoscope system according to the first embodiment, and thus, here, only differences from the first embodiment will be described and detailed description of the rest will be omitted.

As described above, in the endoscope system 1 according to the first embodiment, the waveguide 41 that serves as a signal transmission path that allows propagation of millimeter waves/submillimeter waves is provided so as to extend inside a part, on the proximal end side of the image pickup unit 20 provided in the distal end rigid portion 10 of the insertion portion 6, of the insertion portion 6, the operation portion 7 and the universal cord 8 and reaches the video processor 3.

On the other hand, in an endoscope system 201 according to the present third embodiment, a waveguide that serves as described above is provided so as to extend from the proximal end side of an image pickup unit 20 provided in a distal end rigid portion 10 of an insertion portion 6 to an operation portion 7, and in a part on the proximal side of the operation portion 7, signals are transmitted via optical fibers.

As illustrated in FIG. 9, as in the first embodiment, the endoscope system 201 according to the present third embodiment is what is called an endoscope system for upper digestive tract mainly including: an endoscope 202 including an image pickup section configured to pick up an image of the inside of the body of a subject and output an image signal of the object image; a video processor 203 including an image processing section configured to perform predetermined image processing of the image signal outputted from the image pickup section in the endoscope 202, the video processor 203 being configured to comprehensively control operation of the entire endoscope system 201; a non-illustrated light source apparatus; and a display apparatus.

In the present third embodiment, the endoscope 202 includes an insertion portion 6, an operation portion 7 and a universal cord 8 that are similar to the insertion portion 6, the operation portion 7 and the universal cord 8 in the first embodiment, and the insertion portion 6 includes the distal end rigid portion 10 with the image pickup unit 20 incorporated, the image pickup unit 20 having a configuration that is similar to the image pickup unit 20 in the first embodiment.

Also, as in the first embodiment, a distal end portion of a waveguide 241 configured to allow propagation of millimeter waves or submillimeter waves, the waveguide 241 having a configuration that is similar to the waveguide in the first embodiment, is connected to the proximal end side of a driver IC 23 included in the image pickup unit 20.

In the third embodiment, the waveguide 241 is arranged in such a manner that the distal end side of the waveguide 241 is connected to the driver IC 23 and then is provided so as to extend to a position of a driver IC 71 disposed in the operation portion 7 through the inside of the insertion portion 6.

In the present third embodiment, as illustrated in FIG. 9, inside the operation portion 7, the driver IC 71 for receiving millimeter waves (millimeter wave with image information included) propagating through the waveguide 241, converting the millimeter waves with the image information included into a predetermined optical signal and transmitting the predetermined optical signal to the subsequent stage via an optical fiber is disposed.

More specifically, as in the first embodiment, the driver IC 71 includes: a transmission/reception circuit 72, which is a millimeter wave/submillimeter wave communication circuit formed by what is called an MMIC (monolithic microwave integrated circuit); a transmission/reception antenna 74 for receiving millimeter waves (millimeter waves with image information included) propagating through the waveguide 241, the transmission/reception antenna 74 being connected to the transmission/reception circuit 72; an optical communication circuit 73 configured to generate a signal for predetermined optical communication from image information extracted by the transmission/reception circuit 72; a laser diode (LD) 75 configured to perform photoelectric conversion of the signal generated by the optical communication circuit 73; and a photo diode (PD) 76 configured to perform photoelectric conversion of optical information received from the video processor 203.

Also, in the third embodiment, in the universal cord 8, optical fibers 81, 82 that each serves as a signal transmission path connecting the driver IC 71 in the operation portion 7 and the video processor 203 are disposed.

On the other hand, the video processor 203 includes, in addition to an image processing engine 31 and a power supply circuit 32 that are similar to the processing engine 31 and the power supply circuit 32 in the first embodiment, an optical communication circuit 35 configured to generate a signal for predetermined optical communication, a laser diode (LD) 36 configured to perform photoelectric conversion of the signal generated in the optical communication circuit 35, and a photo diode (PD) 37 configured to perform photoelectric conversion of optical information received from the driver IC 71.

Here, the optical fiber 81 is a signal transmission path connecting the laser diode (LD) 75 and the photo diode (PD) 37, and the optical fiber 82 is a signal transmission path connecting the laser diode (LD) 36 and the photo diode (PD) 76.

Also, as illustrated in FIG. 9, in the present third embodiment, a power wire 242 and a ground wire (GND wire) 243 for power supplied from the power supply circuit 32 in the video processor 203 are disposed in parallel with the waveguide 241 and the optical fibers 81, 82, which are signal transmission paths in the endoscope 202.

As described above, in the present third embodiment, a wired millimeter wave communication path (waveguide) that is less likely to cause breakage of the signal conveyance path is used for a path from the image pickup unit to the operation portion in which deformation such as flexure of the communication path often occurs and an optical fiber communication path, which is advantageous for a long-distance signal transmission, is used for a long-distance path from the operation portion to the video processor in which deformation such as flexure less occurs.

Also, in the present third embodiment, as described above, signal transmission means is optimized according to the usage.

As described above, as in the first embodiment, the present third embodiment enables highly-reliable signal transmission through a wired millimeter wave communication path (waveguide), and in terms of image information transmission speed, enables a high-definition image for up to around full high-definition television to be sent at a practical frame rate.

In addition, as described above, the present third embodiment enables optimization of signal transmission means according to the usage.

### Fourth Embodiment

Next, a fourth embodiment of the present invention will be described.

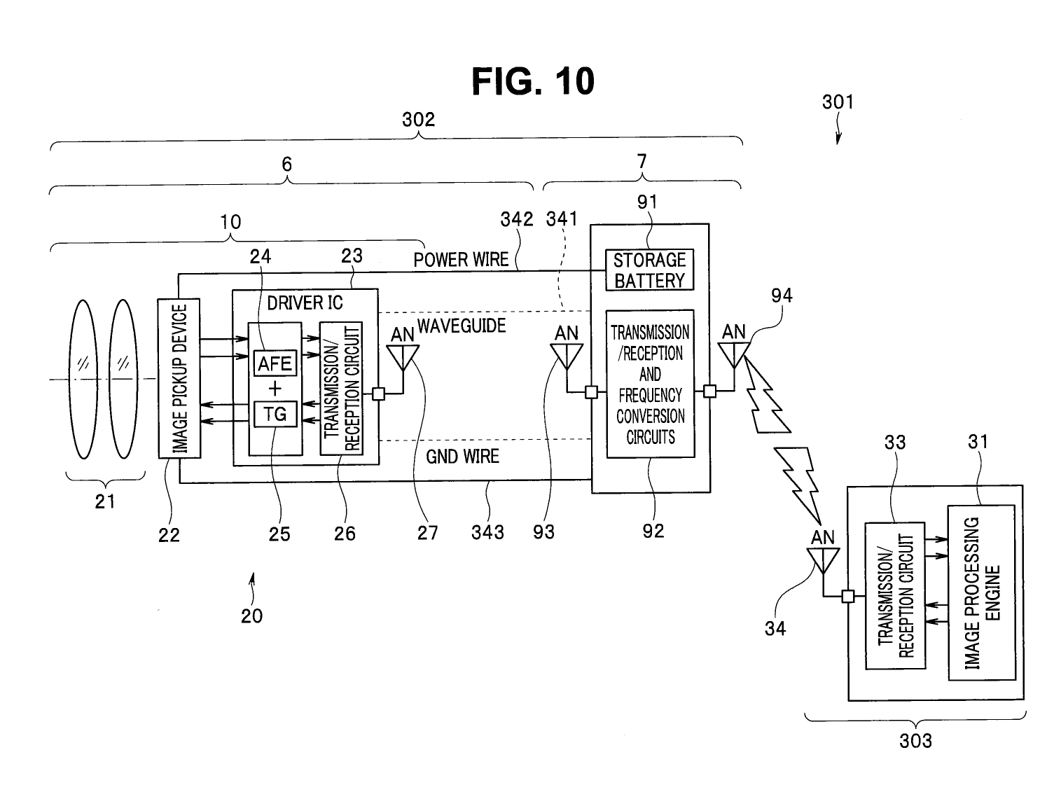

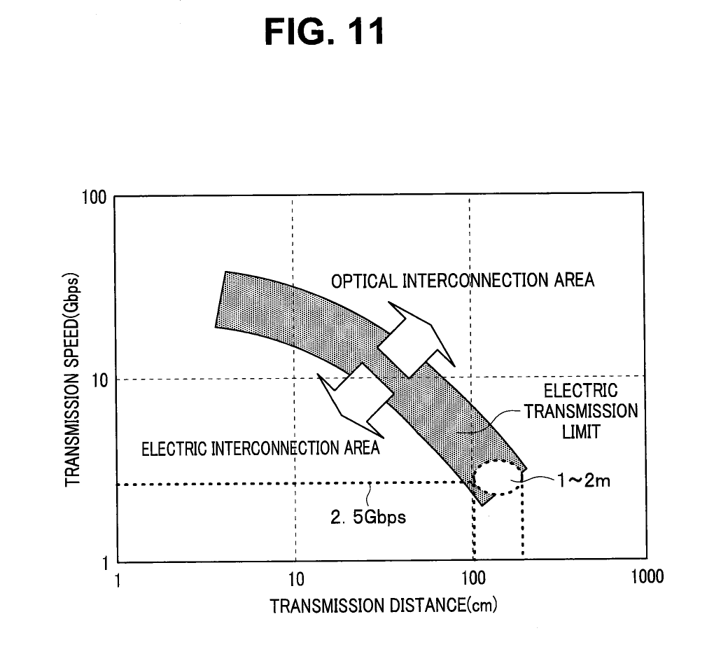

FIG. 10 is a block diagram illustrating a functional configuration of a major part of an endoscope system according to the fourth embodiment of the present invention.

The endoscope system according to the present fourth embodiment is one in which the present invention is applied to what is called a wireless endoscope system, and a configuration of a part from an insertion portion 6 to an operation portion 7 is basically similar to the relevant part in the first embodiment, but signal transmission between the universal cord and the video processor in the first embodiment is performed wirelessly.

Note that description of parts that are in common to the first embodiment such as the configurations of the insertion portion 6 and the image pickup unit 20 will be omitted.

As illustrated in FIG. 10, the endoscope system 301 according to the present fourth embodiment is what is called a wireless endoscope system mainly including: an endoscope 302 including an image pickup section configured to pick up an image of the inside of the body of a subject and output an image signal of the object image; and a video processor 303 including an image processing section configured to perform predetermined image processing of the image signal outputted from the image pickup section, the video processor 303 being configured to perform wireless information communication with the endoscope 302.

In the present fourth embodiment, the endoscope 302 includes a non-illustrated light source in addition to an insertion portion 6 and an operation portion 7 that are similar to the insertion portion 6 and the operation portion 7 in the first embodiment, and the insertion portion 6 includes a distal end rigid portion 10 with an image pickup unit 20 incorporated, the image pickup unit 20 having a configuration that is similar to the configuration of the image pickup unit 20 in the first embodiment.

Also, as in the first embodiment, a distal end portion of a waveguide 341 configured to allow propagation of millimeter waves or submillimeter waves, the waveguide 341 having a configuration that is similar to the waveguide in the first embodiment, is connected to the proximal end side of a driver IC 23 included in the image pickup unit 20.

In the present fourth embodiment, the waveguide 341 is arranged in such a manner that the distal end side of the waveguide 341 is connected to the driver IC 23 and then is provided as to extend to reach a position of the operation portion 7 through the inside of the insertion portion 6.

In the present fourth embodiment, as illustrated in FIG. 10, inside the operation portion 7, a transmission/reception section 92 for receiving millimeter waves (millimeter waves with image information included) propagating through the waveguide 341, converting the millimeter waves with the image information included into a predetermined radio signal and transmitting the predetermined radio signal to the video processor 303 wirelessly is disposed.

More specifically, as in the first embodiment, the transmission/reception section 92 includes a frequency conversion circuit for wireless communication in addition to a transmission/reception circuit, which is a millimeter wave/submillimeter wave communication circuit formed by what is called an MMIC (monolithic microwave integrated circuit).

Furthermore, the transmission/reception section 92 includes a transmission/reception antenna 93 for receiving millimeter waves (millimeter waves with image information included) propagating through the waveguide 341 and an antenna 94 for wireless communication. Also, inside the operation portion 7, a storage battery 91, which serves as a power source for the entire endoscope 302, is provided.

On the other hand, the video processor 303 includes a transmission/reception circuit 33 for wireless communication with the transmission/reception section 92 on the endoscope 302 side and an antenna 34 in addition to an image processing engine 31 that is similar to the image processing engine in the first embodiment.

Also, as illustrated in FIG. 10, in the present fourth embodiment, a power wire 342 and a ground wire (GND wire) 343 for power supplied from the storage battery 91 are disposed in parallel with the waveguide 341, which serves as the signal transmission path, in the endoscope 302.

As described above, in the present fourth embodiment, in the wireless endoscope system, a wired millimeter wave communication path (waveguide) that is less likely to cause breakage of the signal conveyance path is used for a part from the image pickup unit to the operation portion in which deformation such as flexure often occurs, and a wireless communication path, which is advantageous for a long-distance signal transmission, is used for a long-distance path from the operation portion to the video processor, and signal transmission means is thus optimized according to the usage as in the third embodiment.

As described above, according to the present fourth embodiment, what is called a wireless endoscope system also can exert effects that are similar to the effects of the first embodiment for the part from the insertion portion 6 to the operation portion 7, and as in the third embodiment, signal transmission means can be optimized according the usage.

Furthermore, the above embodiment has been described in terms of an example in which a configuration of an endoscope system is employed as an embodiment of the present invention, the present invention is not limited to the example, and the present invention is applicable also to another image pickup system having an image processing function.

### Fifth Embodiment

Next, a fifth embodiment of the present invention will be described.

An endoscope system according to the present fifth embodiment is similar in configuration to the endoscope system according to the first embodiment, and thus, here, only differences from the first embodiment will be described and detailed description of the rest will be omitted.

As described above, the present invention proposes a new signal transmission method using a waveguide configured to allow transmission of millimeter waves or submillimeter waves (radio waves having a frequency of approximately 30 to 600 GHz) instead of a lead wire-used signal transmission method and an optical fiber-used signal transmission method, which have conventionally been used as signal transmission methods connecting an image pickup section in an endoscope and an image processing section in a video processor.

Also, in the endoscope system 1 according to the first embodiment, the waveguide 41 is formed by providing metal plating to the periphery of a polystyrene resin (dielectric material having a permittivity of approximately 2.3 and a dielectric tangent of approximately 0.0002) and an inner diameter of the metal plating surface is set as 1.4 mm and a frequency of radio waves used for transmission of image information is set as approximately 180 GHz (wavelength inside the waveguide is approximately 1.1 mm).

Furthermore, in the first embodiment, the shape and the dimension of the waveguide 41 have been described on the assumption that, for example, a round waveguide tube is used as a waveguide configured to allow transmission of millimeter waves or submillimeter waves.

On the other hand, in an endoscope system according to the present fifth embodiment, a configuration of a waveguide is different from the configuration of the waveguide in the first embodiment, that is, the configuration of the waveguide is more specifically indicated.

The configuration of the waveguide in the present fifth embodiment will more specifically be described below.

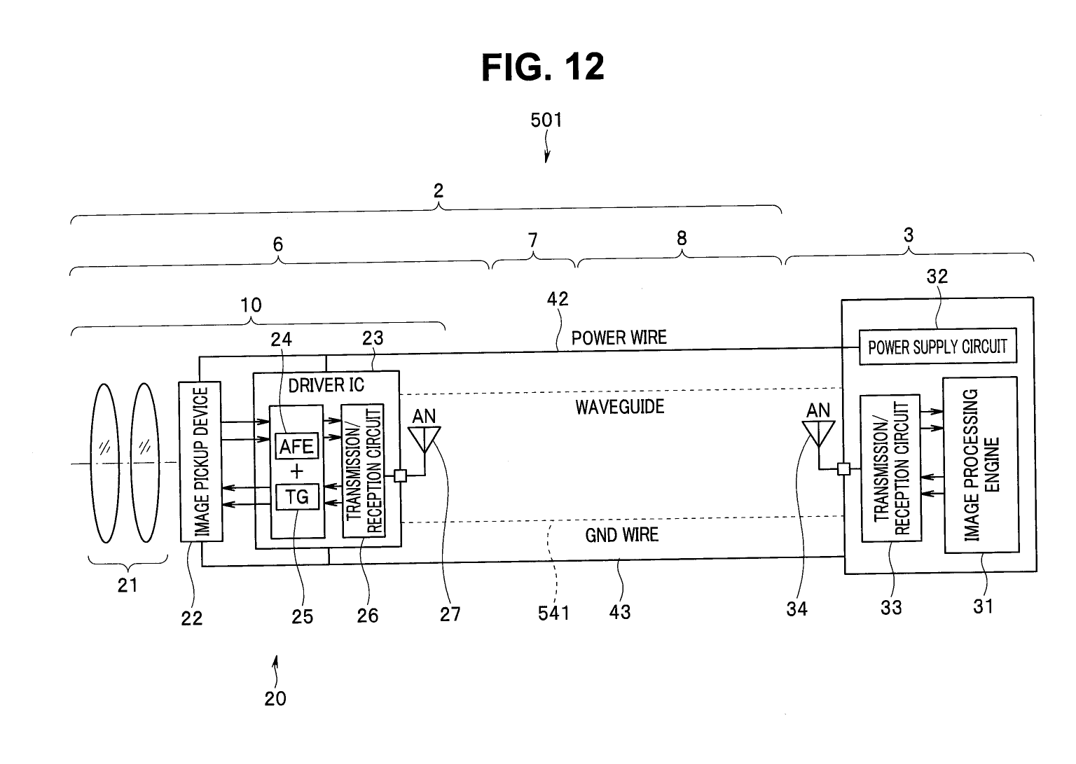

FIG. 12 is a block diagram illustrating a functional configuration of a major part of an endoscope system according to the fifth embodiment of the present invention.

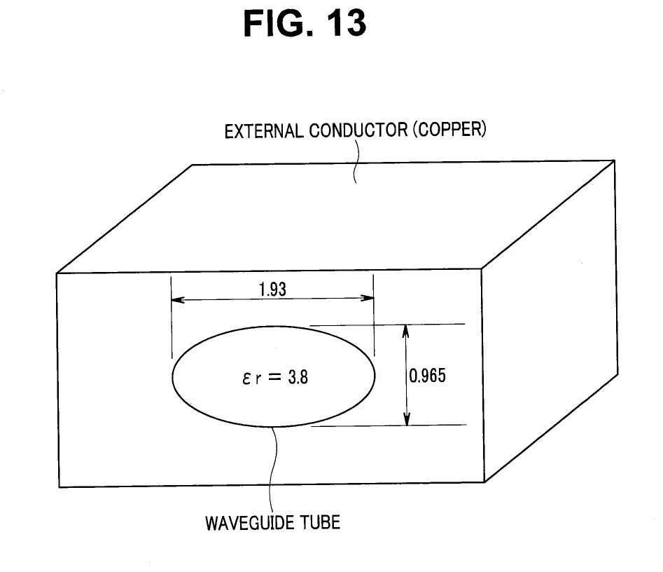

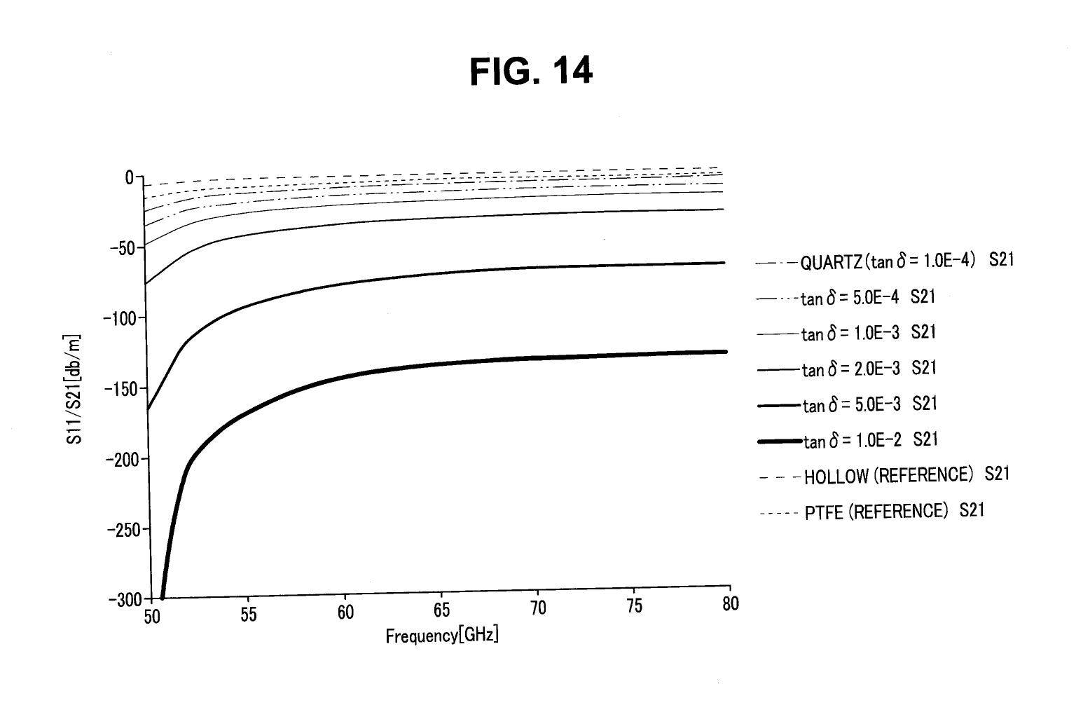

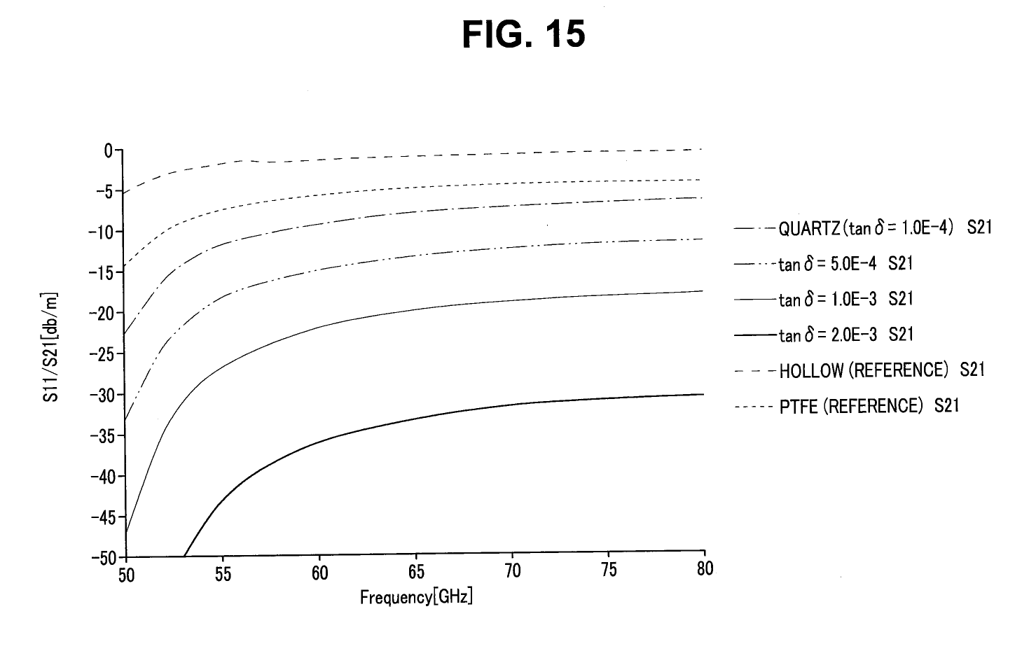

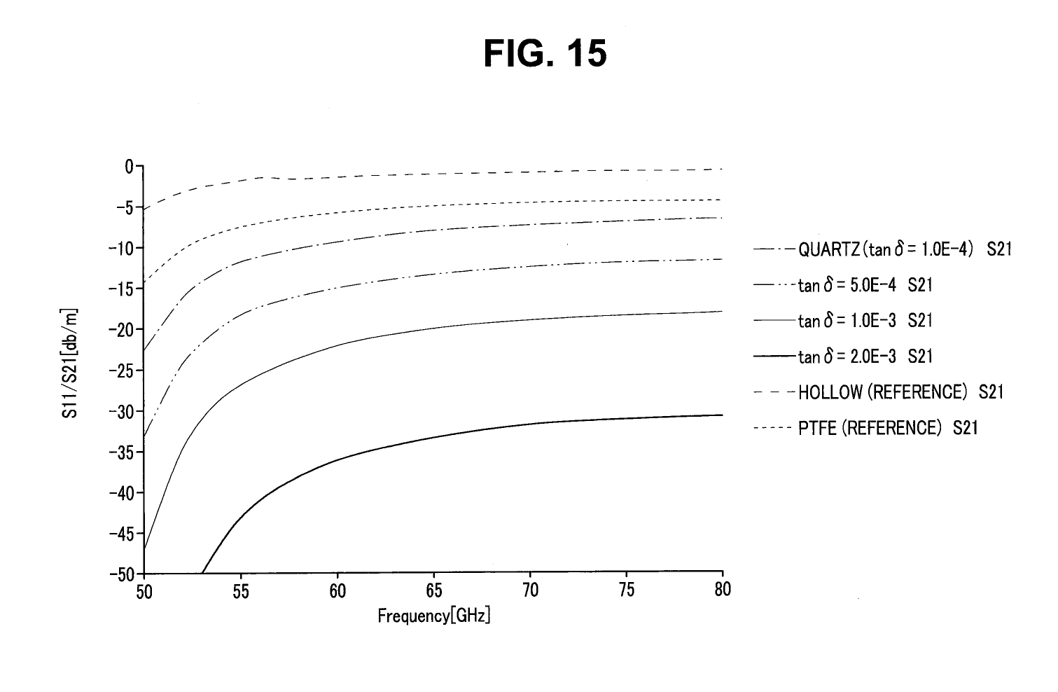

Also, FIGS. 13, 14 and 15 are diagrams illustrating simulations of a waveguide tube employed in the endoscope system according to the fifth embodiment: FIG. 13 is a diagram illustrating a form of a simulation model modeling a waveguide tube employed in the endoscope system according to the fifth embodiment; FIG. 14 is a diagram indicating results of simulations of a dielectric loss of a dielectric material in the simulation model in FIG. 13; and FIG. 15 is an enlarged view of a major part of the simulation results.

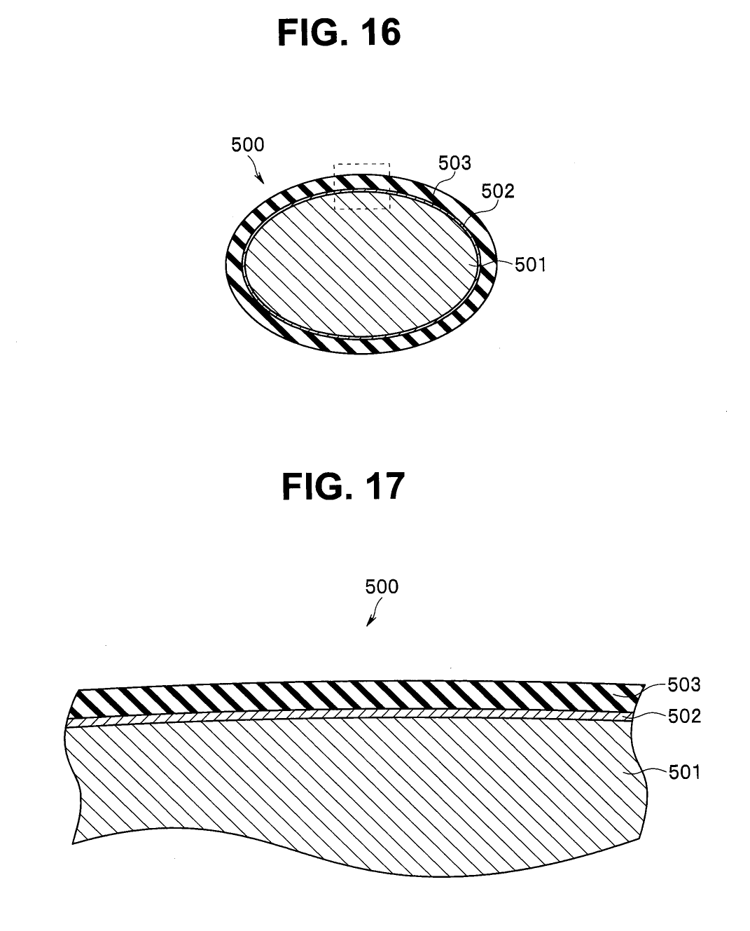

Furthermore, FIG. 16 is a cross-sectional view illustrating a cross-section of the waveguide tube employed in the endoscope system according to the fifth embodiment, and FIG. 17 is an enlarged view illustrating a major part of the cross-section of the waveguide tube.

In an endoscope system 501 according to the present fifth embodiment, as in the first embodiment, in a waveguide 541 (see FIG. 12), a substantially-entire signal transmission path from an image pickup unit 20 to an image processing engine 31 is configured by a waveguide tube configured to allow propagation of millimeter waves or submillimeter waves.

### <Configuration of Waveguide Tube in Fifth Embodiment>

Also, in the present fifth embodiment, the waveguide 541 is configured by a waveguide tube 500 including a dielectric material 501 extending so as to have a permittivity that is uniform in a longitudinal direction and a metal layer 502 extending continuously in the longitudinal direction and covering the outer periphery of the dielectric material, and a dielectric tangent tan δ of the dielectric material 501 has a value that is smaller than 10⁻³ (see FIG. 16). Note that a configuration of the waveguide 541 and the waveguide tube 500 will be described in detail later.

In addition, in the present embodiment, “permittivity is uniform” means that the permittivity is uniform from the perspective of a dimension on the order of a wavelength of radio waves (millimeter waves or submillimeter waves) propagating through the inside of the waveguide tube.

In other words, a permittivity distribution provided by a structure having a dimension that is different from the wavelength order by one or two or more digits does not affect radio waves propagating through the inside of the waveguide tube, and thus, in the present embodiment, the expression “permittivity is uniform” is used in consideration of such point.

As described later, in the present embodiment, it is assumed that a dielectric material obtained by mixing a crystalline material into a resin material is used;

however, in this case, the mixed dielectric material is much smaller than the wavelength. Consequently, neither a difference in permittivity between the resin material and the crystalline material nor the microscopic structure affects radio waves inside the waveguide tube, and only an averaged permittivity affects a transmission characteristic.

A configuration of the waveguide tube in the waveguide 541 of the present fifth embodiment will be described in more detail, but before the description, a critical meaning of the dielectric tangent tan δ of the dielectric material in the waveguide tube 500 having a value that is smaller than 10⁻³ will be described. More specifically, a critical meaning of “the product of the square root of a relative permittivity ∈ᵣ and a dielectric tangent tan δ is smaller than 2×10⁻³” will be described.

As already described, the present invention is applicable to millimeter waves and submillimeter waves, and the effects can be exerted with a transmission line having a thickness of no more than ϕ6 mm; however, for the present invention, as a result of a detailed study of the status of the technology at the time of the present invention being made, the present inventors first derived the following conditions as requirements for a waveguide tube that is highly useful in internal communication in an endoscope.

(1) Having an outer diameter of no more than ϕ2 mm
(2) Transmission loss not exceeding 20 dB per meter

Here, condition (1) is a physical restriction condition for incorporating the waveguide tube in an endoscope, which is derived from configurations of endoscope products at the time of the present invention being made. Here, as described above, an insertion portion and a universal cord of an endoscope usually each have an outer diameter dimension of no more than around 10 mm because of respective purposes of the insertion portion and the universal cord.

In other words, in consideration of many incorporated components such as a light guide for illuminating an observation part, inner structures such as wires for bending a bending portion 9 (see FIG. 1), a water feeding tube for cleaning an objective lens, an air feeding tube for facilitating observation (for example, inflating a stomach) and a treatment instrument channel configured to allow insertion of a treatment instrument for treatment of the observation part being included inside an insertion portion and a universal cord, as condition (1), a specific numerical value that is highly likely to be allowed for a transmission line at the time of the present invention being made is set.

Likewise, condition (2) is set in consideration of a restriction due to a capability of a transmission/reception circuit at the time of the present invention being made (in order to obtain a sufficiently-low bit error rate, a transmission loss of around no more than 20 dB is required) and a minimum available length (approximately 1 m) of an endoscope.

Also, in view of the status of development of the radio wave techniques at the time of the present invention being made, an environment in which 60 GHz from among millimeter wave-band radio waves can easily be used, such as IEEE802.11ad being set as an international standard for next-generation wireless communication, is being put in place (60 Hz is a millimeter radio wave frequency that can most easily be used in consideration of practical use because, e.g., supply of inexpensive wireless communication chips is expected).

In other words, in consideration of the status of these peripheral techniques, the present inventors determined that seeking a waveguide tube technique that can be used with 60 GHz from among millimeter radio waves is a shortcut to practical use on the premise that a waveguide tube configured to allow propagation of millimeter radio waves is applied to communication inside an endoscope.

At the time of the present invention being made, techniques for making up to 300 GHz available for general devices is being developed, and in near future, up to 300 GHz may become available. If such stage is reached, a thinner waveguide tube can be used by increasing the frequency, but the present inventors believe that even at such stage, the present invention would not lose the value and can widely be used.

As a result of a diligent study in consideration of these requirements, the present inventors found that in order to achieve an outer diameter of no more than ϕ2 mm at a frequency of 60 GHz, obtainment of an effect of shortening a wavelength of electromagnetic waves by disposition of a dielectric material inside a waveguide tube (wavelength f of electromagnetic waves is shortened in a medium having a relative permittivity ∈ᵣ in inverse proportion to the square root of ∈ᵣ) is effective.

In addition, as a result of repeatedly making prototypes, the present inventors found that in a transmission loss where a dielectric material is disposed inside a waveguide tube, a dielectric loss of the dielectric material (loss caused by the dielectric material) is dominant. Furthermore, from a theoretical study, the inventors found that the amount of the loss largely depends on “the product of the square root of the relative permittivity ∈ᵣ and the dielectric tangent tan δ”.

Furthermore, the present inventors conducted a study using an electromagnetic field simulator assuming a waveguide tube having an elliptical shape in cross-section and having a long diameter of around no more than 2 mm at a frequency of 60 GHz (∈ᵣ=3.8) (see FIG. 13) and obtained the simulation results indicated in FIGS. 14 and 15.

Here, from the simulation results (see FIGS. 14 and 15), it was found that a dielectric loss of around 20 dB per meter is obtained where the dielectric tangent tan δ is around 1.0×10⁻³.

Also, it was found that if the dielectric tangent tan δ exceeds such value, the dielectric loss rapidly increases, resulting in the dielectric loss amount being no longer an allowable loss amount.

In other words, the present inventors clarified, as a result of the study, that a loss amount largely depends on “the product of the square root of a relative permittivity ∈ᵣ and a dielectric tangent tan δ”, and also clarified, as a result of the above simulation results (if the dielectric tangent tan δ exceeds around 1.0×10⁻³ at a relative permittivity ∈ᵣ of 3.8, the loss amount exceeds an allowable loss amount (20 dB/m)), that for a waveguide tube used for internal communication in an endoscope, as a dielectric material inside the waveguide, the product of the square root of the relative permittivity ∈ᵣ and the dielectric tangent tan δ needs to be roughly no more than 2.0×10⁻³ (since the square root of ∈ᵣ=3.8 is 1.95).

Although the fact is a result derived based on the model in FIG. 3, the relationship among a loss amount, a relative permittivity and a dielectric tangent is one derived from the theoretical study, and is applicable to waveguide tubes extending so as to have a permittivity that is uniform in a longitudinal direction in general.

From the elements clarified here, the present inventors sought a material, the product of the square root of a relative permittivity ∈ᵣ and a dielectric tangent tan δ of which is substantially no more than 2.0×10⁻³.

Here, as a result of the present inventors diligently conducting the seeking, it was found that in resin materials, a fluorine resin, e.g., polytetrafluoroethylene (PTFE), and non-polar plastics such as polyethylene, polypropylene and polystyrene meet the condition and are highly likely to be able to be used for a waveguide tube in the present embodiment.

Furthermore, the present inventors performed screening of the non-polar plastics to find one that can be used for an endoscope system according to the present embodiment. As a result, it was found that from among the non-polar plastics, only fluorine resins, e.g., polytetrafluoroethylene (PTFE) have a temperature resistance necessary for an endoscope (roughly no less than 140° C. in a medical endoscope and roughly no less than 120° C. in an industrial endoscope) and thus are particularly highly useful from among the non-polar plastics.

In other words, a dielectric material used inside a waveguide or a waveguide tube used in an endoscope system according to the present embodiment is at least partly configured by a material containing a fluorine resin, enabling the waveguide or the waveguide tube to achieve high performance (transmission efficiency).

Also, likewise, it was found that in materials other than resins, several crystalline materials such as silicon dioxide (silica; SiO₂), and aluminum oxide (alumina; Al₂O₃) meet the aforementioned condition.

Furthermore, it was found that from among the crystalline materials, silicon dioxide (silica; SiO₂), aluminum oxide (alumina; Al₂O₃), magnesium oxide (MgO) and boron nitride (BN) are harmless to human bodies and particularly useful for endoscope products.

What is important here is that the crystalline materials have a relative permittivity ∈ᵣ that are larger than relative permittivities of the resin materials and use of such characteristic enables provision of a waveguide tube that is thinner than a waveguide tube only using any of the resin materials having a relative permittivity of approximately 2.0.

In other words, use of a dielectric material containing at least one of silicon dioxide (silica; SiO₂), aluminum oxide (alumina; Al₂O₃), magnesium oxide (MgO) and boron nitride (BN) and having a relative permittivity ∈ᵣ that is larger than 2 enables provision of a thinner waveguide tube configured to allow propagation of millimeter radio waves, the waveguide tube being suitable for an endoscope system.

Here, the crystalline materials are inflexible as they are and thus it is necessary to take some ingenuity such as mixing powdered crystalline material and a resin and charging the mixture into a waveguide tube.

In consideration of the above-described points, in the present fifth embodiment, as illustrated in FIGS. 16 and 17, a waveguide 541 is configured by a waveguide tube 500 including a dielectric material 501 extending so as to have a permittivity that is uniform in a longitudinal direction and a metal layer 502 extending continuously in the longitudinal direction and covering an outer periphery of the dielectric material, and a dielectric tangent tan δ of the dielectric material 501 has a value that is smaller than 10⁻³.

More specifically, for the waveguide tube 500 in the present fifth embodiment, a material obtained by mixing powdered aluminum oxide (Al₂O₃ power; #1 μm) into polytetrafluoroethylene (PTFB) at a predetermined volume ratio was used as the inner dielectric material 501.

Also, as a result of the mixing of the above two types of materials, the resulting material has a relative permittivity ∈ᵣ of approximately 4.0 and a dielectric tangent tan δ of around no more than 2.0×10⁻⁴, and using such material, a linear dielectric material 501 having an elliptical cross-section having a long diameter of 1.88 mm and a short diameter of 0.94 mm was fabricated.

The linear dielectric material 501 is sufficiently flexible because bonds in polytetrafluoroethylene (PTFE) are weakened by the mixing of the aluminum oxide power (Al₂O₃) and the dielectric material 501 has a small wire diameter after all.

Then, the metal layer 502 is disposed on the periphery of the linear dielectric material 501. In the present embodiment, the metal layer 502 is configured by wrapping a polyethylene terephthalate (PET) film with a copper-vapor deposited metal film into a roll shape.

Note that, although in the present embodiment, a metal film obtained by vapor-depositing copper on a resin film such as a polyethylene terephthalate (PET) film is employed as the metal layer 502, the present invention is not limited to the example, and a metal film formed by vapor-depositing gold, silver or aluminum on a resin film may be employed.

Also, a thin silicone rubber tape 503 is disposed on an outer layer of the metal layer 502. The tape 503 externally holds the outer layer of the metal layer 502 and thus forms an external conductor (protection layer).

The waveguide tube 500 in the present embodiment having such configuration as described above enabled provision of a flexible waveguide tube having an elliptical cross-section having a long diameter of approximately 2.0 mm and a short diameter of approximately 1.1 mm, the flexible waveguide tube causing a sufficiently-low loss (approximately 13 dB/m) at a frequency of 60 GHz.

### Sixth Embodiment

Next, a sixth embodiment of the present invention will be described.

An endoscope system according to the present sixth embodiment is basically similar in configuration to the endoscope system according to the first embodiment, and thus, here, only differences from the first embodiment will be described and detailed description of the rest will be omitted.

The endoscope system according to the present sixth embodiment is different from the first embodiment in terms of a configuration of a waveguide, that is, as in the fifth embodiment, the configuration of the waveguide is more specifically indicated.

The configuration of the waveguide in the present sixth embodiment will more specifically be described below.

The dielectric material bar inside such waveguide tube as described above has an elliptical shape in cross-section and has directionality in ease of flexing. In other words, the dielectric material bar can easily be flexed in a short diameter direction of the ellipse but may be difficult to smoothly flex in a long diameter direction of the ellipse.

In view of such point, the present applicant provides a flexible waveguide tube having improved directionality in ease of flexing while a transmission characteristic being maintained.

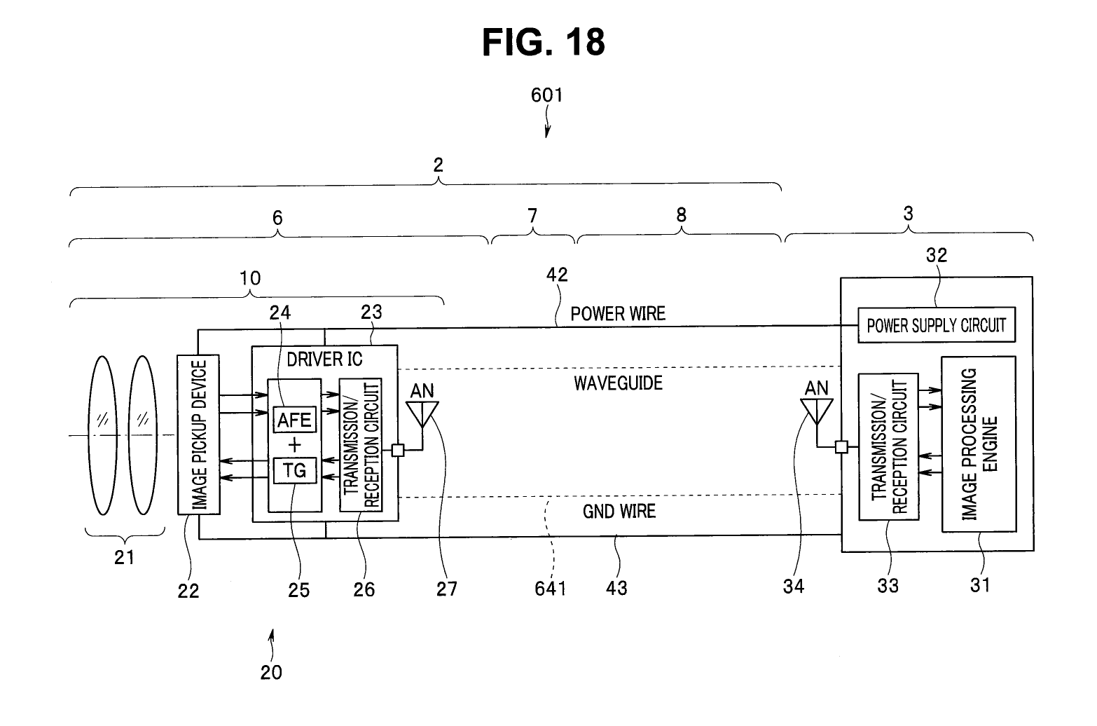

FIG. 18 is a block diagram illustrating a functional configuration of a major part of an endoscope system according to the sixth embodiment of the present invention.

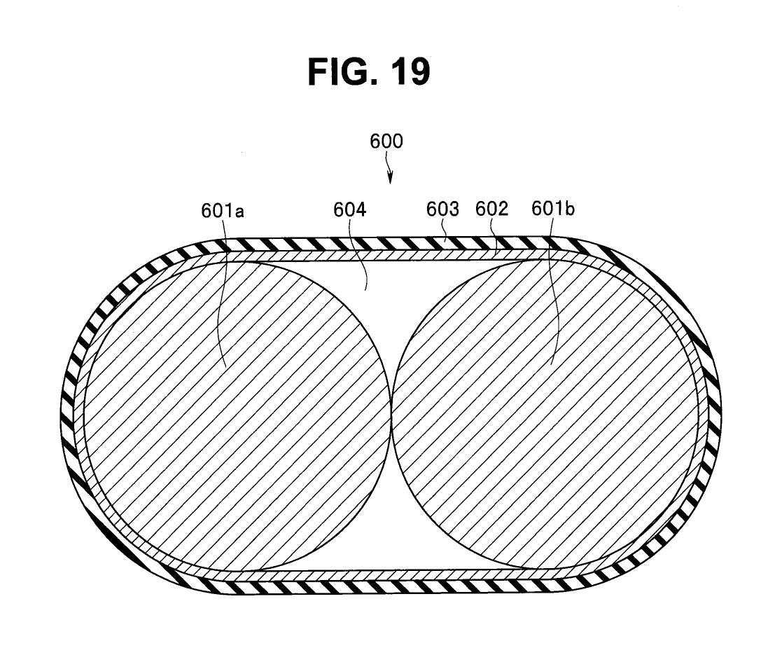

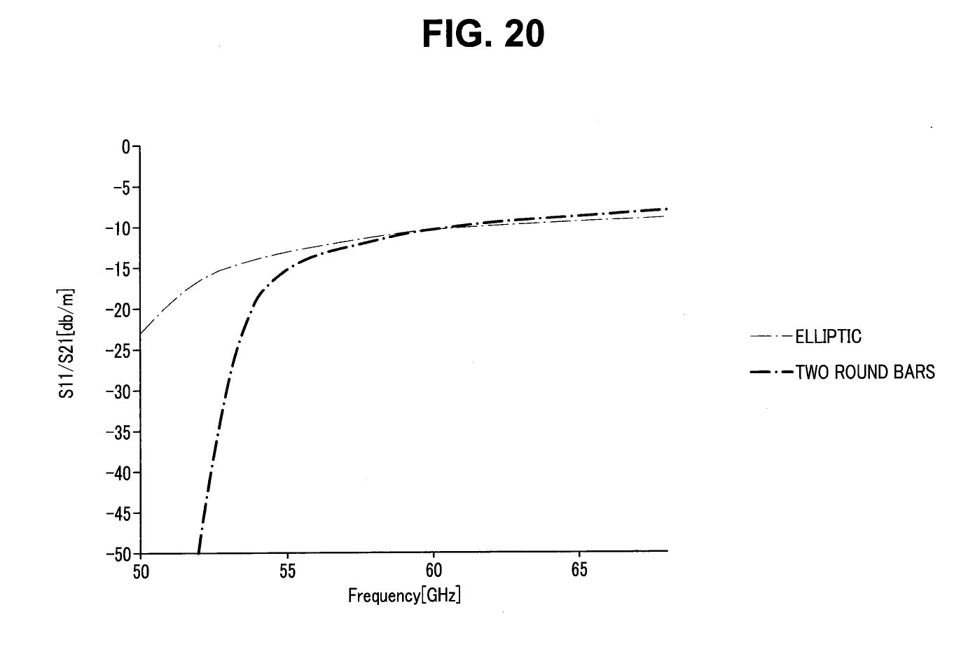

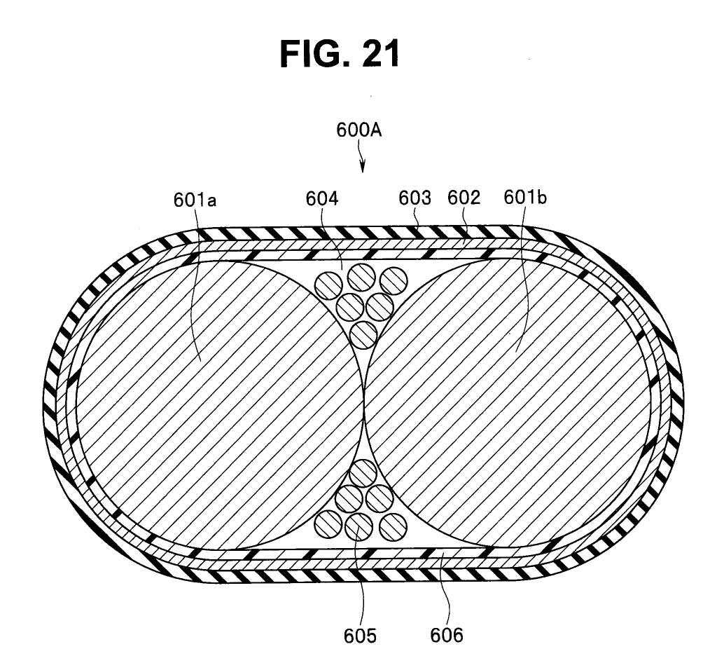

Also, FIG. 19 is a cross-sectional view illustrating a cross-section of a waveguide tube according to the sixth embodiment, and FIG. 20 is a diagram indicating results of simulations of a dielectric loss of dielectric materials in a waveguide tube according to the sixth embodiment. Furthermore, FIG. 21 is a cross-sectional view illustrating a cross-section of a modification of the waveguide tube according to the sixth embodiment.

In an endoscope system 601 according to the present sixth embodiment, as in the first embodiment, a substantially-entire signal transmission path from an image pickup unit 20 to an image processing engine 31 in a waveguide 641 (see FIG. 18) is configured by a waveguide tube that allows transmission of millimeter waves or submillimeter waves.

### <Configuration of Waveguide Tube in Sixth Embodiment>

As illustrated in FIG. 19, in a waveguide tube 600 in the present sixth embodiment, two flexible dielectric materials 601a, 601b each having a round shape in cross-section are used as inner dielectric materials.

In other words, the flexible waveguide tube 600 in the sixth embodiment is a waveguide tube for transmission of radio waves, the waveguide tube including an area surrounded by a metal layer 602, the area having a required length, in which two flexible dielectric materials 601a, 601b each having a round cross-section continuing in a longitudinal direction are disposed as core materials.

In the present embodiment, the two dielectric materials 601a, 601b are each formed by, for example, PFA (tetrafluoroethylene-perfluoroalkylvinylether copolymer).

Also, in the flexible waveguide tube 600 in the present embodiment, the metal layer 602 is disposed on the periphery of the two dielectric materials 601a, 601b. As in the fifth embodiment, the metal layer 602 is configured by wrapping a metal film with a copper-vapor deposited on a polyethylene terephthalate (PET) film into a roll shape.

Here, in the present embodiment, also, as the metal layer 602, a metal film formed by vapor-depositing copper on a resin film such as a polyethylene terephthalate (PET) film is employed; however, the present invention is not limited to the example, and a metal film formed by vapor-depositing gold, silver or aluminum on a resin film may be employed.

Then, in the present embodiment, a cross-sectional shape of the area surrounded by the metal layer 602 is defined by the two flexible dielectric materials 601a, 601b each having a round shape in cross-section.

Also, a thin silicone rubber tape 603 is disposed on an outer layer of the metal layer 602. The tape 603 externally holds the outer layer of the metal layer 602 and thus forms an outer conductor (protection layer).

In the flexible waveguide tube 600 in the sixth embodiment, a space portion 604 is formed between the two dielectric materials 601a, 601b.

In the waveguide tube 600 in the present sixth embodiment configured as above, the core materials are configured by the flexible dielectric materials 601a, 601b each having a round shape in cross-section, that is, two round bars formed using PFA (tetrafluoroethylene-perfluoroalkylvinylether copolymer), and thus, even if an external flexing force is applied in a longitudinal direction of the cross-section, the waveguide tube 600 is sufficiently easily flexed because each of the inner dielectric materials 601a, 601b having a round shape in cross-section and the inner materials slide on each other.

Also, as can be seen from the numerical simulation results indicated in FIG. 20, a transmission characteristic of the waveguide tube 600 itself is comparable to the transmission characteristic of the above-described waveguide tube 500 in the fifth embodiment having an elliptical cross-sectional shape.

FIG. 21 is a cross-sectional view illustrating a cross-section of a modification of the waveguide tube according to the sixth embodiment.

In the waveguide tube 600A according to the modification, for example, string-like portions 605 configured using PFA (tetrafluoroethylene-perfluoroalkylvinylether copolymer) are inserted in the space portion 604 formed between the two round bar-like dielectric materials 601a, 601b in the waveguide tube 600.

Furthermore, in the waveguide tube 600A according to the modification, a film-like portion 606 configured using PFA (tetrafluoroethylene-perfluoroalkylvinylether copolymer) is disposed between the dielectric materials 601a, 601b and the metal layer 602.

As described above, the waveguide tube 600A according to the modification enables further improvement in transmission characteristic without losing flexibility, by the insertion of the PFA string-like portions 605 to the space portion 604.

Furthermore, the disposition of the PFA film-like portion 606 between the dielectric materials 601a, 601b and the metal layer 602 improves slidability of the inner materials and thus contributes to further flexibility enhancement.

The present invention is not limited to the above-described embodiments, and various modifications, alterations and the like are possible without departing from the spirit of the present invention.

What is claimed is:

1. An endoscope system comprising: an insertion portion in which an image pickup unit configured to pick up an image of an object to be examined and generate a video signal is disposed in a distal end; a video processing section configured to process the video signal generated by the image pickup unit; and a signal transmission path connecting the image pickup unit and the video processing section,
wherein at least a part of the signal transmission path is a waveguide configured to allow propagation of a millimeter wave or a submillimeter wave, and signal transmission is performed by the waveguide.

2. The endoscope system according to claim 1, wherein the waveguide includes a waveguide tube including a dielectric material extending so that a permittivity is uniform in a longitudinal direction, and a metal layer continuously extending in the longitudinal direction and covering an outer periphery of the dielectric material.

3. The endoscope system according to claim 2, wherein a dielectric tangent tan δ of the dielectric material is a value that is smaller than 10⁻³.

4. The endoscope system according to claim 2, wherein the dielectric material includes a material at least partially including a fluorine resin.

5. The endoscope system according to claim 2, wherein the dielectric material includes at least one of silicon dioxide, aluminum oxide, magnesium oxide and boron nitride, and a relative permittivity ∈ᵣ of the dielectric material is larger than 2.

6. The endoscope system according to claim 2, wherein the dielectric material includes two core materials each with a round-shaped cross-section.

7. The endoscope system according to claim 2, wherein the metal layer includes a metal film including any one of gold, silver, copper and aluminum, and a resin film.

8. The endoscope system according to claim 1, wherein a solid-state image pickup device including a number of pixels, the number being equal to or exceeding two million pixels, is included in the image pickup unit.

9. The endoscope system according to claim 1, further comprising a millimeter wave/submillimeter wave communication circuit including an MMIC (monolithic microwave integrated circuit).

10. The endoscope system according to claim 9, wherein:
the insertion portion includes a distal end portion in which the image pickup unit is disposed and a bending portion for changing a direction of the distal end portion;
the MMIC (monolithic microwave integrated circuit) is disposed in the distal end portion; and
the waveguide is disposed at least in the bending portion.

11. The endoscope system according to claim 2, wherein the dielectric material includes a resin material and a crystalline material having a relative permittivity that is larger than a relative permittivity of the resin material.

12. The endoscope system according to claim 6, wherein the dielectric material includes a string member including a tetrafluoroethylene-perfluoroalkylvinylether copolymer between the two core materials.
